(19) United States
(12) Patent Application Publication
(10) Pub. No.: US 2017/0240539 A1
(43) Pub. Date: Aug. 24, 2017

(54) Preparation and Use of Cyclic Sulfonamide Derivatives as PAR-1 Receptor Antagonists

(71) Applicant: MERCK SHARP & DOHME CORP., Rahway, NJ (US)

(72) Inventors: Andrew W. Stamford, Chatham, NJ (US); Zhi-Qiang Yang, Westfield, NJ (US); Milana Maletic, Summit, NJ (US); Senlin Cai, Shanghai (CN); Xiaobang Duan, Shanghai (CN); Jianhua Cao, Shanghai (CN)

(73) Assignee: MERCK SHARP & DOHME CORP., Rahway, NJ (US)

(21) Appl. No.: 15/518,804

(22) PCT Filed: Oct. 9, 2015
(86) PCT No.: PCT/US15/54803

§ 371 Date: Apr. 13, 2017

(30) Foreign Application Priority Data
Oct. 15, 2014 (CN) PCT/CN2014/088655

(51) Int. Cl. C07D 417/06; C07D 417/14

(52) U.S. Cl. CPC C07D 417/06; C07D 417/14

(57) ABSTRACT

The present invention relates to cyclic sulfonamide derivatives of Formula (I) or a pharmaceutically acceptable salt thereof.

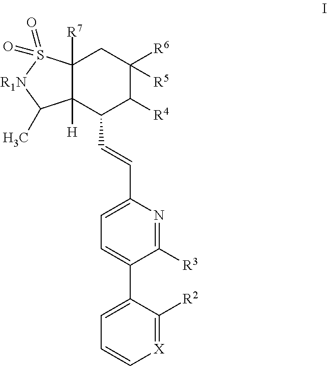

### FIELD OF THE INVENTION

The present invention relates to cyclic sulfonamide derivatives, which are useful as protease activated receptor-1 (PAR-1) antagonists. PAR-1 receptors are also known in the art as thrombin receptors and thus PAR-1 antagonists are also referred to as thrombin receptor antagonists (TRA). As PAR-1 antagonists, the compounds described herein may have utility in treating disease states such as acute coronary syndrome (ACS) (including unstable angina, non-ST-segment elevation [NSTE], myocardial infarction [MI], and ST segment-elevation myocardial infarction [STEMI]), secondary prevention of myocardial infarction or thrombotic stroke (secondary prevention) or peripheral artery disease (PAD), which is also known in the art as peripheral vascular disease. Also described herein are pharmaceutical compositions comprising the cyclic sulfonamide derivatives as well as processes for their preparation.

### REFERENCE TO SEQUENCE LISTING

The official copy of the Sequence Listing is submitted concurrently with the specification as an ASCII formatted text file via EFS-Web, with a file name of “23823PSP.txt”, a creation date of Oct. 6, 2014 and a size of 1760 bytes. The Sequence Listing filed via EFS-Web is part of the specification and incorporated in its entirety by reference herein.

### BACKGROUND OF THE INVENTION

Thrombin is known to have a variety of activities in different cell types. PAR-1 receptors are known to be present in such cell types as human platelets, vascular smooth muscle cells, endothelial cells and fibroblasts. The art indicates that PAR-1 receptor antagonists would be expected to be useful in the treatment of thrombotic, inflammatory, atherosclerotic and fibroproliferative disorders, as well as other disorders in which thrombin and its receptor play a pathological role.

Thrombin receptor antagonist peptides have been identified based on structure-activity studies involving substitutions of amino acids on thrombin receptors. In Bernatowicz et al., J. Med. Chem., 39 (1996), p. 4879-4887, tetra- and pentapeptides are disclosed as being potent thrombin receptor antagonists, for example N-trans-cinnamoyl-p-fluoroPhe-p-guanidinoPhe-Leu-Arg-NH₂ (SEQ ID NO: 1) and N-trans-cinnamoyl-p-fluoroPhe-p-guanidinoPhe-Leu-Arg-Arg-NH₂ (SEQ ID NO: 2). Peptide thrombin receptor antagonists are also disclosed in WO 94/03479.

Substituted bi- and tricyclic thrombin receptors antagonists are known in the art to treat thrombin receptor mediated disorders such as thrombosis, atherosclerosis, restenosis, hypertension, angina pectoris, angiogenesis related disorders, arrhythmia, a cardiovascular or circulatory disease or condition, heart failure, ACS, myocardial infarction, glomerulonephritis, thrombotic stroke, thromboembolytic stroke, PAD, deep vein thrombosis, venous thromboembolism, a cardiovascular disease associated with hormone replacement therapy, disseminated intravascular coagulation syndrome and cerebral infarction. U.S. Pat. No. 6,645,987 and U.S. Pat. No. 6,894,065 disclose PAR-1 receptor antagonists of the structure:

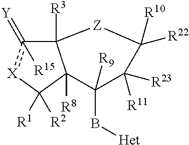

where R¹⁰ may be groups such as H, alkyl, haloalkyl, hydroxyl, etc. and R²² may be groups such as H, optionally substituted alkyl, hydroxyl, etc. Other known substituted thrombin receptor antagonists are disclosed in WO2001/96330, U.S. Pat. No. 6,063,847, U.S. Pat. No. 6,326,380, U.S. Pat. No. 7,037,920, U.S. Pat. No. 7,488,742, U.S. Pat. No. 7,713,999, U.S. Pat. No. 7,442,712, U.S. Pat. No. 7,488,752, U.S. Pat. Nos. 7,776,889, 7,888,369, U.S. Pat. No. 8,003,803 and U.S. Pat. No. 8,022,088, US 2008/0090830 and Chackalamannil et al., J. Med. Chem., 49 (2006), p. 5389. A PAR-1 receptor antagonist that exhibits good thrombin receptor antagonist activity (potency) and selectivity is vorapaxar, tradename ZONTIVITY (Merck & Co., Inc.), which has the following structure:

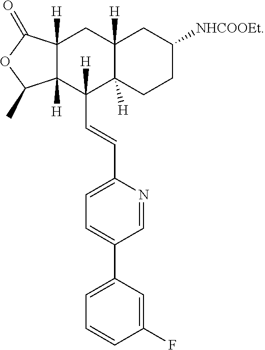

This compound is disclosed in U.S. Pat. No. 7,304,048. A crystalline form of the bisulfate salt of vorapaxar is disclosed in U.S. Pat. No. 7,235,567.

WO2011/162,562 to LG Life Sciences LTD. describes a series of [6+5] fused bicycle derivatives of the general structure:

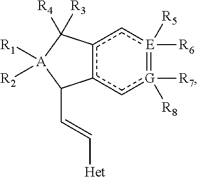

where R₅ and R₆ are inter alia both fluoro groups, as inhibitors of the PAR-1 receptor. The compounds are taught to be useful in the treatment and prevention of thrombus, platelet aggregation, atherosclerosis, restenosis, blood coagulation, hypertension, arrhythmia, angina pectoris, heart failure, inflammation and cancer when used alone or with other cardiovascular agents.

WO2011/28420 and WO2011/28421, both to Sanofi-Aventis, disclose compounds that are reported to be PAR-1 receptor antagonists. The compounds disclosed in WO2011/28420 are pyridyl-vinyl pyrazoloquinolines derivatives and have the following general structure:

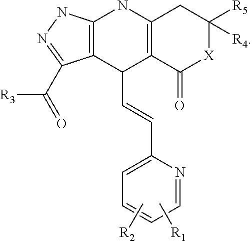

WO2011/28421 discloses tryicyclic pyridyl-vinyl-pyrrole derivatives of the following general structure:

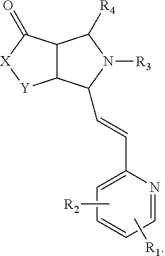

PCT/US13/027383 to Merck Sharp and Dohme, Corp. discloses bicyclic himbacine derivatives of the following general structure

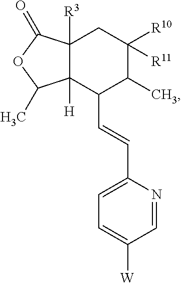

where R¹⁰ and R¹¹ may both be fluoro groups. These compounds are PAR-1 receptor antagonists.

Applicants discovered that the compounds described herein act as inhibitors of PAR-1 receptor and, based upon their structure, the compounds might be expected to be useful in treating disease states associated with the inhibition of this receptor.

There is a need for new compounds, formulations, treatment and therapies to treat diseases associated with the PAR-1 receptor. Moreover, there is a need to develop therapeutics that exhibit improved therapeutic profiles; for example, desirable half-life or reduced unintended effects, such as not causing drug induced (acquired) long QT syndrome, which potentially can be fatal, or reduced drug-drug interactions (DDIs). DDIs are potentially undesirable as they can reduce the therapeutic effectiveness of an agent or increase the incidence of unintended effects associated with the drug. It is, therefore, an object of this invention to provide compounds useful in the treatment, prevention or amelioration of such diseases or disorders with possible improved therapeutic profiles.

### SUMMARY OF THE INVENTION

Described herein are PAR-1 receptor antagonists of cyclic sulfonamide derivatives of Formula I:

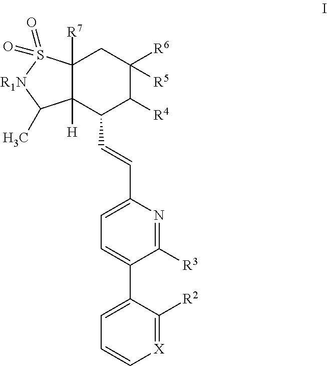

or a pharmaceutically acceptable salt thereof, wherein:

X is —N—, —CH or —CR⁸;

R¹ is selected from the group consisting of hydrogen, C₁-C₆alkyl, COC₁-C₆alkyl, COOH and COOC₁-C₆alkyl, wherein any C₁-C₆alkyl can be unsubstituted or substituted with one or more substituents independently selected from the group consisting of halo, —OH and —CN;

R² is selected from the group consisting of halo, C₁-C₆alkyl, OC₁-C₆alkyl, —CN and hydrogen wherein when X is —CR⁸, R² is hydrogen;

R³ is selected from the group consisting of hydrogen, halo and C₁-C₆alkyl;

R⁴ is selected from the group consisting of hydrogen, halo and C₁-C₆alkyl;

R⁵ is selected from the group consisting of hydrogen, halo and C₁-C₆alkyl;

R⁶ is selected from the group consisting of hydrogen, halo and C₁-C₆alkyl;

R⁷ is selected from the group consisting of hydrogen and C₁-C₆alkyl, wherein C₁-C₆alkyl can be unsubstituted or substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN; and
R⁸ is selected from the group consisting of halo and C₁-C₆alkyl.

Another aspect of the present invention is pharmaceutical compositions comprising a therapeutically effective amount of at least one compound of Formula I or a pharmaceutically acceptable salt thereof and a pharmaceutically acceptable carrier.

Another aspect of the present invention is pharmaceutical compositions comprising a therapeutically effective amount of at least one compound of Formula I or a pharmaceutically acceptable salt thereof, at least one additional cardiovascular agent and a pharmaceutically acceptable carrier.

Another aspect of the present invention is the possible treatment or prevention of one or more disease states associated with inhibiting the PAR-1 receptor by administering an effective amount of a compound of Formula I or a pharmaceutically acceptable salt thereof to a patient in need thereof.

Another aspect of the present invention is a method of inhibiting platelet aggregation comprising administering to a mammal an effective amount of a compound of Formula I or a pharmaceutically acceptable salt thereof.

It is further contemplated that the combination of the invention could be provided as a kit comprising in a single package at least one compound of Formula I or a pharmaceutically acceptable salt thereof in a pharmaceutical composition, and at least one separate pharmaceutical composition, such as, for example a separate pharmaceutical composition comprising a cardiovascular agent.

The compounds of the present invention can potentially be useful in the treatment, amelioration or prevention of one or more conditions associated with inhibiting the PAR-1 receptor by administering at least one compound of Formula I or a pharmaceutically acceptable salt thereof to a mammal in need of such treatment. Conditions that could potentially be treated or prevented by inhibiting the PAR-1 receptor include thrombosis, atherosclerosis, restenosis, hypertension, angina pectoris, angiogenesis related disorders, arrhythmia, a cardiovascular or circulatory disease or condition, heart failure, ACS, myocardial infarction, glomerulonephritis, thrombotic stroke, thromboembolytic stroke, PAD, deep vein thrombosis, venous thromboembolism, a cardiovascular disease associated with hormone replacement therapy, disseminated intravascular coagulation syndrome and cerebral infarction.

Another embodiment is the possible treatment, amelioration or prevention of ACS, secondary prevention of myocardial infarction or stroke, urgent coronary revascularization, or PAD by administering at least one compound of Formula I or a pharmaceutically acceptable salt thereof to a mammal in need of such treatment.

Another embodiment of this invention is in the possible treatment, amelioration or prevention of one or more conditions associated with cardiopulmonary bypass surgery (CPB) by administering an effective amount of at least one compound of Formula I or a pharmaceutically acceptable salt thereof to a subject undergoing said CPB surgery. CPB surgery includes coronary artery bypass graft surgery (CABG), cardiac valve repair and replacement surgery, and pericardial and aortic repair surgeries. The conditions associated with CABG include bleeding, thrombotic vascular events (such as thrombosis or restenosis), vein graft failure, artery graft failure, atherosclerosis, angina pectoris, myocardial ischemia, acute coronary syndrome, myocardial infarction, heart failure, arrhythmia, hypertension, transient ischemic attack, cerebral function impairment, thromboembolic stroke, cerebral ischemia, cerebral infarction, thrombophlebitis, deep vein thrombosis and PAD.

Another embodiment of the present invention is the possible use of a compound of Formula I or a pharmaceutically acceptable salt thereof for the manufacture of a medicament for the treatment, amelioration or prevention of one or more conditions associated with inhibiting the PAR-1 receptor in a patient.

### DESCRIPTION OF THE INVENTION

As used above, and throughout this disclosure, the following terms, unless otherwise indicated, shall be understood to have the following meanings:

“Patient” or “subject” includes both humans and animals.

“Mammal” means humans and other mammalian animals.

“C₁-C₆alkyl” means an aliphatic hydrocarbon group which may be straight or branched and comprising about 1 to about 6 carbon atoms in the chain. Branched means that one or more lower alkyl groups such as methyl, ethyl or propyl, are attached to a linear alkyl chain.

“Alkoxy” or “OC₁-C₆alkyl” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Halo” refers to fluorine, chlorine, bromine or iodine radicals. Preferred are fluoro, chloro or bromo, and more preferred are fluoro and chloro.

“Halogen” means fluorine, chlorine, bromine, or iodine. Non-limiting examples include fluorine or chlorine.

The term “isolated” or “in isolated form” for a compound refers to the physical state of said compound after being isolated from a synthetic process or natural source or combination thereof.

The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

When a functional group in a compound is termed “protected,” this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

“Effective amount” or “therapeutically effective amount” is meant to describe an amount of compound or a composition of the present invention effective as PAR-1 or thrombin receptor antagonists, thereby producing the desired therapeutic, ameliorative, inhibitory or preventative effect.

Described herein are compounds of Formula I:

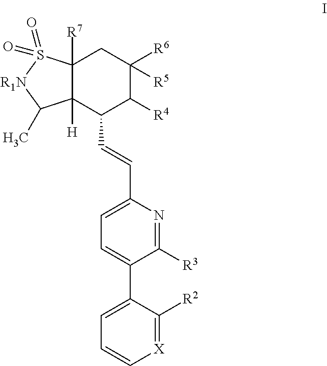

or a pharmaceutically acceptable salt thereof, wherein X is —N—, —CH or —CR⁸; R¹ is selected from the group consisting of hydrogen, C₁-C₆alkyl, COC₁-C₆alkyl, COOH and COOC₁-C₆alkyl, wherein any C₁-C₆alkyl can be unsubstituted or substituted with one or more substituents independently selected from the group consisting of halo, —OH and —CN; R² is selected from the group consisting of halo, C₁-C₆alkyl, OC₁-C₆alkyl, —CN and hydrogen, wherein when X is —CR⁸, R² is hydrogen; R³ is selected from the group consisting of hydrogen, halo and C₁-C₆alkyl; R⁴ is selected from the group consisting of hydrogen, halo and C₁-C₆alkyl; R⁵ is selected from the group consisting of hydrogen, halo and C₁-C₆alkyl; R⁶ is selected from the group consisting of hydrogen, halo and C₁-C₆alkyl; R⁷ is selected from the group consisting of hydrogen and C₁-C₆alkyl, wherein C₁-C₆alkyl can be unsubstituted or substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN; and R⁸ is selected from the group consisting of halo and C₁-C₆alkyl.

In certain embodiments, the compounds described herein have the following stereochemistry as shown in a compound of Formula Ia:

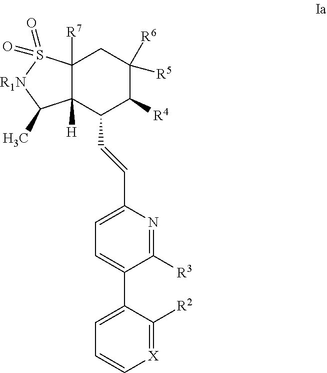

or a pharmaceutically acceptable salt thereof, wherein X is —N—, —CH or —CR⁸; R¹ is selected from the group consisting of hydrogen, C₁-C₆alkyl, COC₁-C₆alkyl, COOH and COOC₁-C₆alkyl, wherein any C₁-C₆alkyl can be unsubstituted or substituted with one or more substituents independently selected from the group consisting of halo, —OH and —CN; R² is selected from the group consisting of halo, C₁-C₆alkyl, OC₁-C₆alkyl, —CN and hydrogen, wherein when X is —CR⁸, R² is hydrogen; R³ is selected from the group consisting of hydrogen, halo and C₁-C₆alkyl; R⁴ is selected from the group consisting of hydrogen, halo and C₁-C₆alkyl; R⁵ is selected from the group consisting of hydrogen, halo and C₁-C₆alkyl; R⁶ is selected from the group consisting of hydrogen, halo and C₁-C₆alkyl; R⁷ is selected from the group consisting of hydrogen and C₁-C₆alkyl, wherein C₁-C₆alkyl can be unsubstituted or substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN; and R⁸ is selected from the group consisting of halo and C₁-C₆alkyl.

In certain embodiments, the compounds described herein have the following stereochemistry as shown in a compound of Formula Ib:

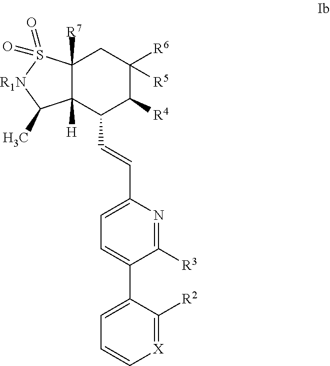

or a pharmaceutically acceptable salt thereof, wherein X is —N—, —CH or —CR⁸; R¹ is selected from the group consisting of hydrogen, C₁-C₆alkyl, COC₁-C₆alkyl, COOH and COOC₁-C₆alkyl, wherein any C₁-C₆alkyl can be unsubstituted or substituted with one or more substituents independently selected from the group consisting of halo, —OH and —CN; R² is selected from the group consisting of halo, C₁-C₆alkyl, OC₁-C₆alkyl, —CN and hydrogen, wherein when X is —CR⁸, R² is hydrogen; R³ is selected from the group consisting of hydrogen, halo and C₁-C₆alkyl; R⁴ is selected from the group consisting of hydrogen, halo and C₁-C₆alkyl; R⁵ is selected from the group consisting of hydrogen, halo and C₁-C₆alkyl; R⁶ is selected from the group consisting of hydrogen, halo and C₁-C₆alkyl; R⁷ is selected from the group consisting of hydrogen and C₁-C₆alkyl, wherein C₁-C₆alkyl can be unsubstituted or substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN; and R⁸ is selected from the group consisting of halo and C₁-C₆alkyl.

In other embodiments, the compounds described herein have the following stereochemistry as shown in a compound of Formula Ic:

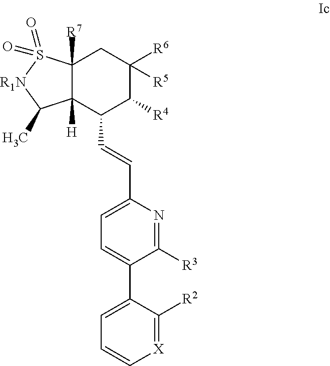

or a pharmaceutically acceptable salt thereof, wherein X is —N—, —CH or —CR⁸; R¹ is selected from the group consisting of hydrogen, C₁-C₆alkyl, COC₁-C₆alkyl, COOH and COOC₁-C₆alkyl, wherein any C₁-C₆alkyl can be unsubstituted or substituted with one or more substituents independently selected from the group consisting of halo, —OH and —CN; R² is selected from the group consisting of halo, C₁-C₆alkyl, OC₁-C₆alkyl, —CN and hydrogen, wherein when X is —CR⁸, R² is hydrogen; R³ is selected from the group consisting of hydrogen, halo and C₁-C₆alkyl; R⁴ is selected from the group consisting of hydrogen, halo and C₁-C₆alkyl; R⁵ is selected from the group consisting of hydrogen, halo and C₁-C₆alkyl; R⁶ is selected from the group consisting of hydrogen, halo and C₁-C₆alkyl; R⁷ is selected from the group consisting of hydrogen and C₁-C₆alkyl, wherein C₁-C₆alkyl can be unsubstituted or substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN; and R⁸ is selected from the group consisting of halo or C₁-C₆alkyl.

In certain embodiments, the compounds described herein have the following stereochemistry as shown in a compound of Formula Id:

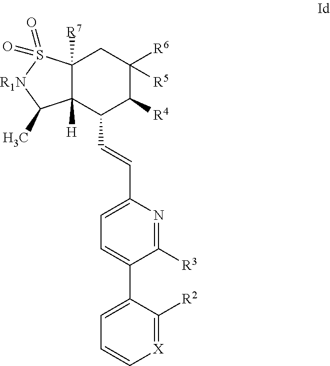

or a pharmaceutically acceptable salt thereof, wherein X is —N—, —CH or —CR⁸; R¹ is selected from the group consisting of hydrogen, C₁-C₆alkyl, COC₁-C₆alkyl, COOH and COOC₁-C₆alkyl, wherein any C₁-C₆alkyl can be unsubstituted or substituted with one or more substituents independently selected from the group consisting of halo, —OH and —CN; R² is selected from the group consisting of halo, C₁-C₆alkyl, OC₁-C₆alkyl, —CN and hydrogen, wherein when X is —CR⁸, R² is hydrogen; R³ is selected from the group consisting of hydrogen, halo and C₁-C₆alkyl; R⁴ is selected from the group consisting of hydrogen, halo and C₁-C₆alkyl; R⁵ is selected from the group consisting of hydrogen, halo and C₁-C₆alkyl; R⁶ is selected from the group consisting of hydrogen, halo and C₁-C₆alkyl; R⁷ is selected from the group consisting of hydrogen and C₁-C₆alkyl, wherein C₁-C₆alkyl can be unsubstituted or substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN; and R⁸ is selected from the group consisting of halo and C₁-C₆alkyl.

In other embodiments, the compounds described herein have the following stereochemistry as shown in a compound of Formula Ie:

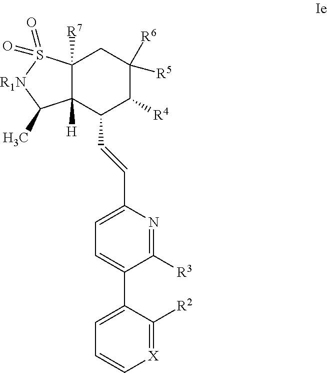

or a pharmaceutically acceptable salt thereof, wherein X is —N—, —CH or —CR⁸; R¹ is selected from the group consisting of hydrogen, C₁-C₆alkyl, COC₁-C₆alkyl, COOH and COOC₁-C₆alkyl, wherein any C₁-C₆alkyl can be unsubstituted or substituted with one or more substituents independently selected from the group consisting of halo, —OH and —CN; R² is selected from the group consisting of halo, C₁-C₆alkyl, OC₁-C₆alkyl, —CN and hydrogen, wherein when X is —CR⁸, R² is hydrogen; R³ is selected from the group consisting of hydrogen, halo and C₁-C₆alkyl; R⁴ is selected from the group consisting of hydrogen, halo and C₁-C₆alkyl; R⁵ is selected from the group consisting of hydrogen, halo and C₁-C₆alkyl; R⁶ is selected from the group consisting of hydrogen, halo and C₁-C₆alkyl; R⁷ is selected from the group consisting of hydrogen and C₁-C₆alkyl, wherein C₁-C₆alkyl can be unsubstituted or substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN; and R⁸ is selected from the group consisting of halo or C₁-C₆alkyl.

With regard to the compounds described herein, X can be —N—, —CH or —CR⁸. In certain embodiments of the compounds described herein, X is —N—. In other embodiments of the compounds described herein, X is —CH. In still other embodiments of the compounds described herein, X is —CR⁸.

With regard to the compounds described herein R¹ can be hydrogen, C₁-C₆alkyl, COC₁-C₆alkyl, COOH or COOC₁-C₆alkyl. In certain embodiments of the compounds described herein, R¹ is hydrogen. In other embodiments of the compounds described herein, R¹ is C₁-C₆alkyl. In still other embodiments of the compounds described herein, R¹ is COC₁-C₆alkyl. Suitable alkyls include, but are not limited to, methyl, ethyl and butyl. In yet other embodiments of the compounds described herein, R¹ is COOH. In yet other embodiments of the compounds described herein, R¹ is COOC₁-C₆alkyl. Suitable COOC₁-C₆alkyls include, but are not limited to, COOC(CH₃)₃ and COOCH₂CH₃. In certain embodiments of the compounds described herein, R¹ is hydrogen or COOC₁-C₆alkyl.

R¹ can be unsubstituted or substituted. When R¹ is substituted, R¹ can be substituted with one or more substituents independently selected from the group consisting of halo, —OH and —CN. For example, any C₁-C₆alkyl can be substituted with one or more substituents independently selected from the group consisting of halo, —OH and —CN. In certain embodiments when R¹ is C₁-C₆alkyl, C₁-C₆alkyl can be unsubstituted or substituted with one or more substituents independently selected from the group consisting of halo, —OH and —CN. For example, when R¹ is C₁-C₆alkyl, C₁-C₆alkyl can be substituted with one or more halo substitutents. For example, when R¹ is C₁-C₆alkyl, C₁-C₆alkyl can be substituted with one or more —OH substitutents. For example, when R¹ is C₁-C₆alkyl, C₁-C₆alkyl can be substituted with one or more —CN substitutents.

For example, when R¹ is C₁-C₆alkyl, C₁-C₆alkyl can be substituted with halo. For example, when R¹ is C₁-C₆alkyl, C₁-C₆alkyl can be substituted with —OH. Suitable alcohols include, but are not limited to methanol and ethanol. In one embodiment, R¹ is ethanol. For example, when R¹ is C₁-C₆alkyl, C₁-C₆alkyl can be substituted with —CN.

With regard to the compounds described herein, R² can be halo, C₁-C₆alkyl, OC₁-C₆alkyl, —CN or hydrogen. In certain embodiments of the compounds described herein, R² is halo. Suitable halogen radicals include, but are not limited to, fluoro. In certain embodiments of the compounds described herein, R² is halo. In other embodiments of the compounds described herein, R² is OC₁-C₆alkyl. Suitable alkoxys include, but are not limited to, methoxy. In still other embodiments of the compounds described herein, R² is —CN. In certain embodiments of the compounds described herein, R² is methoxy or —CN.

In certain embodiments of the compounds described herein, R² can be hydrogen. In certain embodiments of the compounds described herein, when X is —CR⁸, R² is hydrogen.

With regard to the compounds described herein, R³ can be hydrogen, halo or C₁-C₆alkyl. In certain embodiments of the compounds described herein, R³ is hydrogen. In certain embodiments of the compounds described herein, R³ is halo. In certain embodiments of the compounds described herein, R³ is C₁-C₆alkyl. In certain embodiments, R³ is hydrogen or methyl.

With regard to the compounds described herein, R⁴ can be hydrogen, halo or C₁-C₆alkyl. In certain embodiments of the compounds described herein, R⁴ is hydrogen. In certain embodiments of the compounds described herein, R⁴ is halo. In certain embodiments of the compounds described herein, R⁴ is C₁-C₆alkyl. Suitable alkyls include, but are not limited to, methyl and ethyl. In certain embodiments, R⁴ is hydrogen or methyl. In certain embodiments, R⁴ is ethyl.

With regard to the compounds described herein, R⁵ can be hydrogen, halo or C₁-C₆alkyl. In certain embodiments of the compounds described herein, R⁵ is hydrogen. In certain embodiments of the compounds described herein, R⁵ is halo. In certain embodiments of the compounds described herein, R⁵ is C₁-C₆alkyl. In certain embodiments, R⁵ is hydrogen or fluoro.

With regard to the compounds described herein, R⁶ can be hydrogen, halo or C₁-C₆alkyl. In certain embodiments of the compounds described herein, R⁶ is hydrogen. In certain embodiments of the compounds described herein, R⁶ is halo. In certain embodiments of the compounds described herein, R⁶ is C₁-C₆alkyl. In certain embodiments, R⁶ is hydrogen or fluoro.

With regard to the compounds described herein, R⁷ can be hydrogen or C₁-C₆alkyl. In certain embodiments of the compounds described herein, R⁷ is hydrogen. In other embodiments of the compounds described herein, R⁷ is C₁-C₆alkyl. In other embodiments of the compounds described herein, R⁷ is hydrogen or methyl.

R⁷ can be unsubstituted or substituted when R⁷ is C₁-C₆alkyl. In certain embodiments of the compounds described herein R⁷ is unsubstituted. In certain embodiments of the compounds described herein R⁷ is substituted when R⁷ is C₁-C₆alkyl. When substituted R⁷ can be substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN. In certain embodiments when R⁷ is C₁-C₆alkyl, C₁-C₆alkyl can be substituted with one or more substituents selected from the group consisting of CONH₂, CONHC₁-C₆alkyl, CON(C₁-C₆alkyl)₂, COC₁-C₆alkyl, COOH, COOC₁-C₆alkyl, halo, —OH and —CN.

For example, when R⁷ is C₁-C₆alkyl, C₁-C₆alkyl can be substituted with one or more CONH₂ substitutents. For example, when R⁷ is C₁-C₆alkyl, C₁-C₆alkyl can be substituted with one or more CONHC₁-C₆alkyl substitutents. For example, when R⁷ is C₁-C₆alkyl, C₁-C₆alkyl can be substituted with one or more CON(C₁-C₆alkyl)₂ substitutents. For example, when R⁷ is C₁-C₆alkyl, C₁-C₆alkyl can be substituted with one or more COC₁-C₆alkyl substitutents. For example, when R⁷ is C₁-C₆alkyl, C₁-C₆alkyl can be substituted with one or more COOH substitutents. For example, when R⁷ is C₁-C₆alkyl, C₁-C₆alkyl can be substituted with one or more COOC₁-C₆alkyl substitutents.

For example, when R⁷ is C₁-C₆alkyl, C₁-C₆alkyl can be substituted with CONH₂. For example, when R⁷ is C₁-C₆alkyl, C₁-C₆alkyl can be substituted with CONHC₁-C₆alkyl. For example, when R⁷ is C₁-C₆alkyl, C₁-C₆alkyl can be substituted with CON(C₁-C₆alkyl)₂. For example, when R⁷ is C₁-C₆alkyl, C₁-C₆alkyl can be substituted with COC₁-C₆alkyl. For example, when R⁷ is C₁-C₆alkyl, C₁-C₆alkyl can be substituted with COOH. For example, when R⁷ is C₁-C₆alkyl, C₁-C₆alkyl can be substituted with COOC₁-C₆alkyl.

For example, when R⁷ is C₁-C₆alkyl, C₁-C₆alkyl can be substituted with one or more halo substitutents. For example, when R⁷ is C₁-C₆alkyl, C₁-C₆alkyl can be substituted with one or more —OH substitutents. For example, when R⁷ is C₁-C₆alkyl, C₁-C₆alkyl can be substituted with one or more —CN substitutents.

For example, when R⁷ is C₁-C₆alkyl, C₁-C₆alkyl can be substituted with halo. For example, when R⁷ is C₁-C₆alkyl, C₁-C₆alkyl can be substituted with —OH. For example, when R⁷ is C₁-C₆alkyl, C₁-C₆alkyl can be substituted with —CN.

With regard to the compounds described herein, R⁸ can be halo or C₁-C₆alkyl. In certain embodiments of the compounds described herein, R⁸ is halo. In certain embodiments of the compounds described herein, R⁸ is C₁-C₆alkyl. In certain embodiments, R⁸ is fluoro.

In certain embodiments of the compounds described herein, the compounds are selected from the group consisting of:

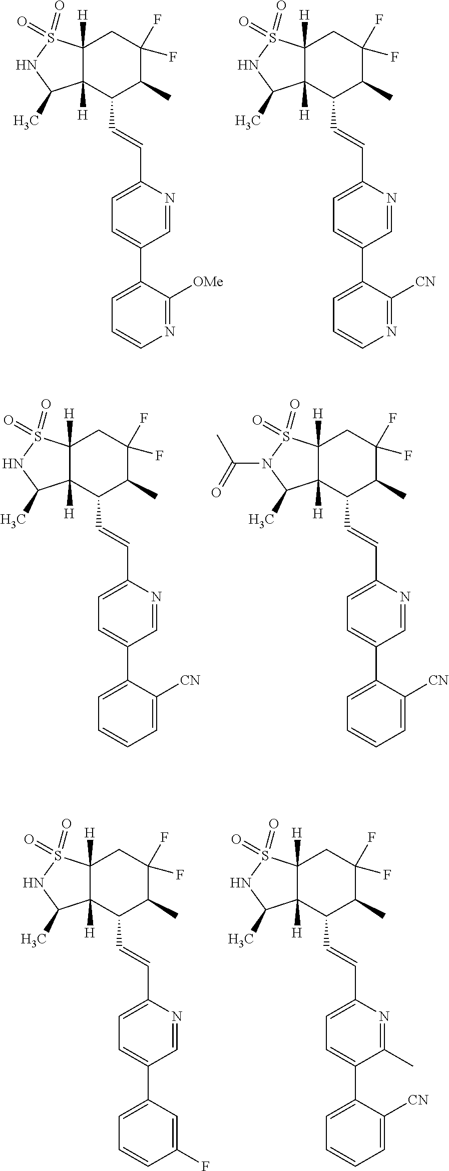

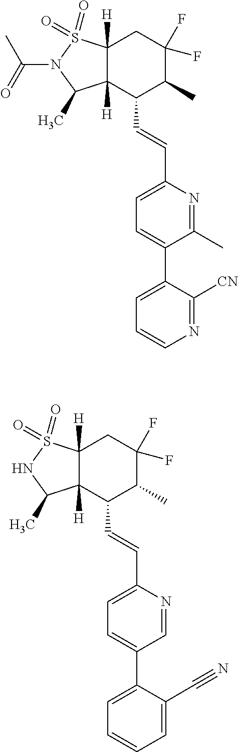 [SMILES: CC(=O)N1[C@H](C)[C@H]2[C@@H](/C=C/c3ccc(-c4cccnc4C#N)c(C)n3)[C@H](C)C(F)(F)C[C@H]2S1(=O)=O ; C[C@@H]1[C@H](/C=C/c2ccc(-c3ccccc3C#N)cn2)[C@H]2[C@@H](CC1(F)F)S(=O)(=O)N[C@@H]2C]

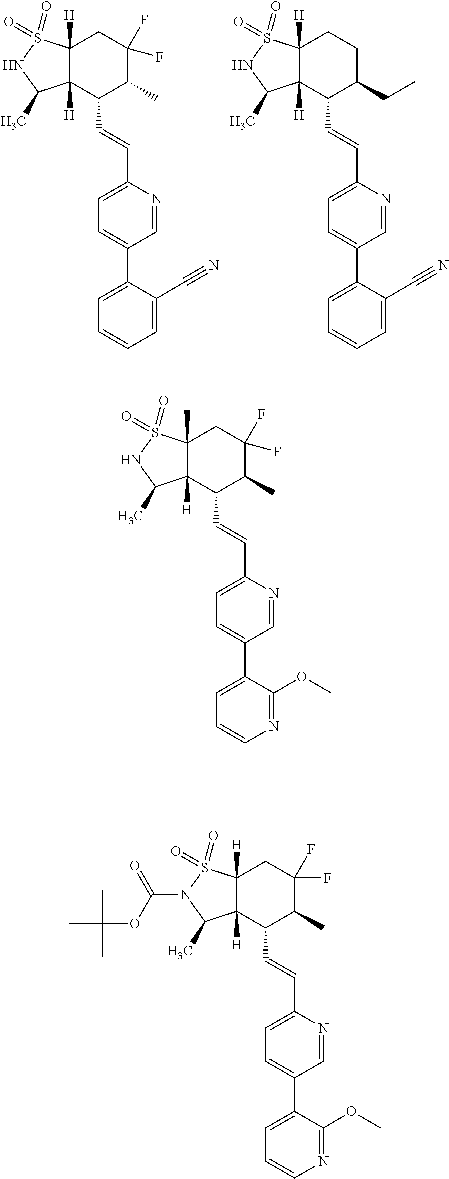 [SMILES: C[C@@H]1[C@H](/C=C/c2ccc(-c3ccccc3C#N)cn2)[C@H]2[C@@H](CC1(F)F)S(=O)(=O)N[C@@H]2C ; CC[C@@H]1CC[C@@H]2[C@H]([C@H]1/C=C/c1ccc(-c3ccccc3C#N)cn1)[C@@H](C)NS2(=O)=O ; COc1ncccc1-c1ccc(/C=C/[C@@H]2[C@@H]3[C@@H](C)NS(=O)(=O)[C@]3(C)CC(F)(F)[C@H]2C)nc1 ; COc1ncccc1-c1ccc(/C=C/[C@@H]2[C@@H]3[C@@H](C)N(C(=O)OC(C)(C)C)S(=O)(=O)[C@@H]3CC(F)(F)[C@H]2C)nc1]

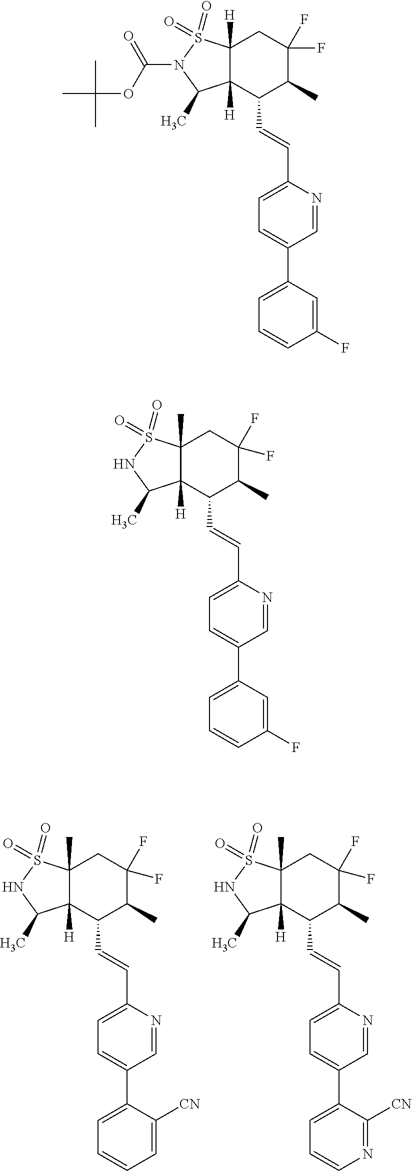 [SMILES: C[C@@H]1[C@H]2[C@@H](/C=C/c3ccc(-c4cccc(F)c4)cn3)[C@H](C)C(F)(F)C[C@H]2S(=O)(=O)N1C(=O)OC(C)(C)C ; C[C@H]1NS(=O)(=O)[C@]2(C)CC(F)(F)[C@@H](C)[C@H](/C=C/c3ccc(-c4cccc(F)c4)cn3)[C@H]12 ; C[C@H]1NS(=O)(=O)[C@]2(C)CC(F)(F)[C@@H](C)[C@H](/C=C/c3ccc(-c4ccccc4C#N)cn3)[C@H]12 ; C[C@H]1NS(=O)(=O)[C@]2(C)CC(F)(F)[C@@H](C)[C@H](/C=C/c3ccc(-c4cccnc4C#N)cn3)[C@H]12]

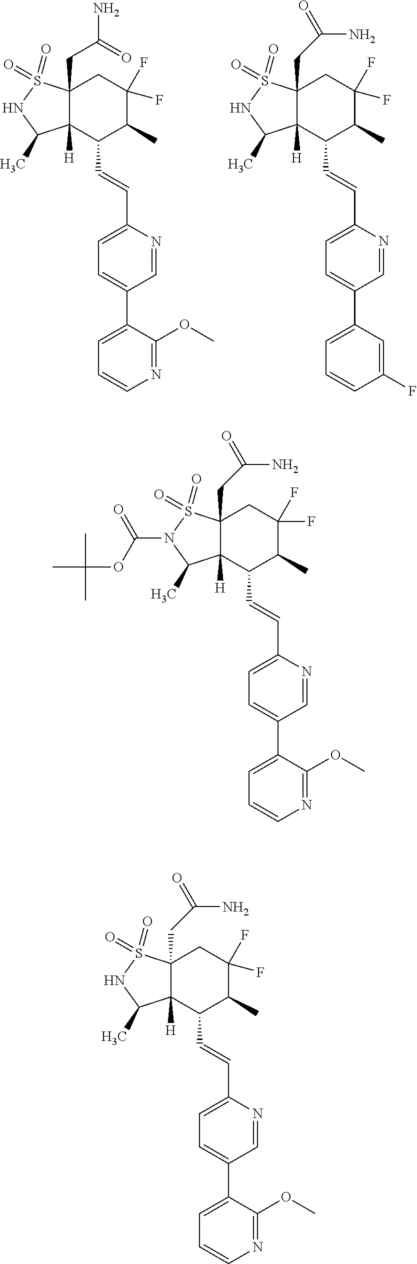 [SMILES: COc1ncccc1-c1ccc(/C=C/[C@@H]2[C@@H]3[C@@H](C)NS(=O)(=O)[C@]3(CC(N)=O)CC(F)(F)[C@H]2C)nc1 ; C[C@H]1NS(=O)(=O)[C@]2(CC(N)=O)CC(F)(F)[C@@H](C)[C@H](/C=C/c3ccc(-c4cccc(F)c4)cn3)[C@H]12 ; COc1ncccc1-c1ccc(/C=C/[C@@H]2[C@@H]3[C@@H](C)N(C(=O)OC(C)(C)C)S(=O)(=O)[C@]3(CC(N)=O)CC(F)(F)[C@H]2C)nc1 ; COc1ncccc1-c1ccc(/C=C/[C@@H]2[C@@H]3[C@@H](C)NS(=O)(=O)[C@@]3(CC(N)=O)CC(F)(F)[C@H]2C)nc1]

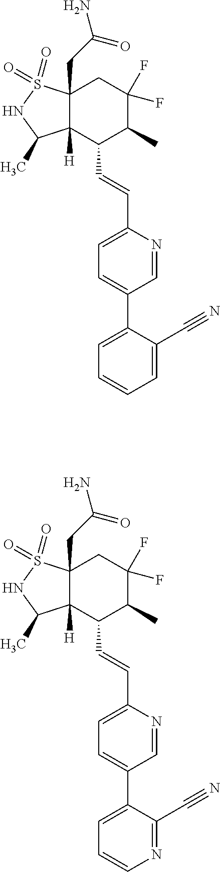 [SMILES: C[C@H]1NS(=O)(=O)[C@]2(CC(N)=O)CC(F)(F)[C@@H](C)[C@H](/C=C/c3ccc(-c4ccccc4C#N)cn3)[C@H]12 ; C[C@H]1NS(=O)(=O)[C@]2(CC(N)=O)CC(F)(F)[C@@H](C)[C@H](/C=C/c3ccc(-c4cccnc4C#N)cn3)[C@H]12]

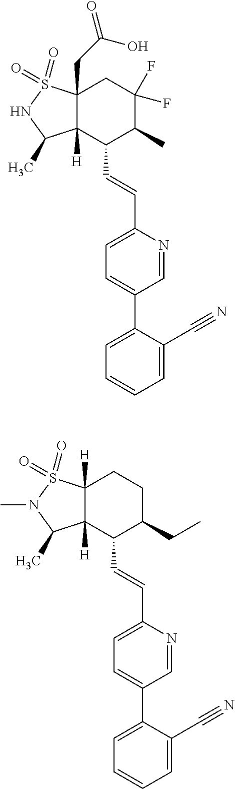 [SMILES: C[C@H]1NS(=O)(=O)[C@]2(CC(=O)O)CC(F)(F)[C@@H](C)[C@H](/C=C/c3ccc(-c4ccccc4C#N)cn3)[C@H]12 ; CC[C@@H]1CC[C@@H]2[C@H]([C@H]1/C=C/c1ccc(-c3ccccc3C#N)cn1)[C@@H](C)N(C)S2(=O)=O]

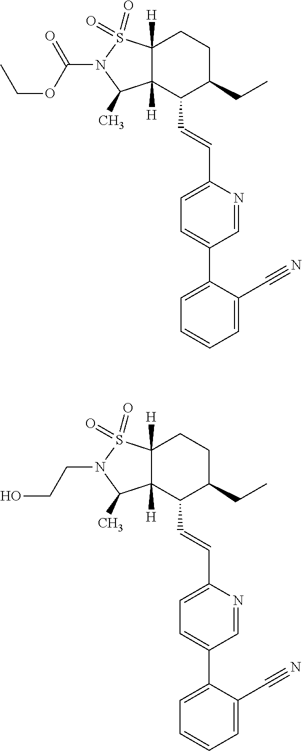 [SMILES: CCOC(=O)N1[C@H](C)[C@H]2[C@@H](/C=C/c3ccc(-c4ccccc4C#N)cn3)[C@H](CC)CC[C@H]2S1(=O)=O ; CC[C@@H]1CC[C@@H]2[C@H]([C@H]1/C=C/c1ccc(-c3ccccc3C#N)cn1)[C@@H](C)N(CCO)S2(=O)=O]

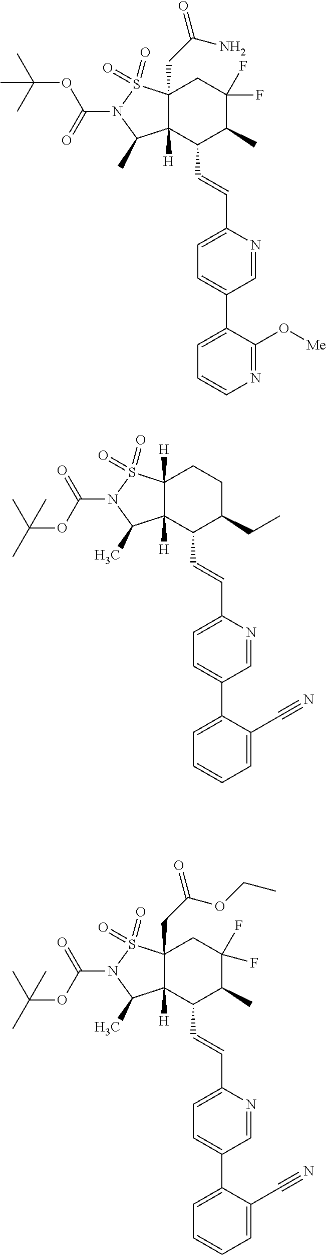 [SMILES: COc1ncccc1-c1ccc(/C=C/[C@@H]2[C@@H]3[C@@H](C)N(C(=O)OC(C)(C)C)S(=O)(=O)[C@@]3(CC(N)=O)CC(F)(F)[C@H]2C)nc1 ; CC[C@@H]1CC[C@@H]2[C@H]([C@H]1/C=C/c1ccc(-c3ccccc3C#N)cn1)[C@@H](C)N(C(=O)OC(C)(C)C)S2(=O)=O ; CCOC(=O)C[C@@]12CC(F)(F)[C@@H](C)[C@H](/C=C/c3ccc(-c4ccccc4C#N)cn3)[C@@H]1[C@@H](C)N(C(=O)OC(C)(C)C)S2(=O)=O]

The compounds of Formula I can form salts, such salts are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), arylalkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

In this application, unless otherwise indicated, whenever there is a structural formula provided, such as those of Formula I, this formula is intended to encompass all forms of a compound such as, for example, any solvates, hydrates, stereoisomers, tautomers, co-crystals, polymorphs etc.

Compounds of Formula I, and salts, solvates, co-crystals and prodrugs thereof, may exist in their tautomeric form (for example, as an amide or imino ether). All such tautomeric forms are contemplated herein as part of the present invention.

Prodrugs, solvates and co-crystals of the compounds of the invention are also contemplated herein. The term “prodrug,” as employed herein, denotes a compound that is a drug precursor which, upon administration to a subject, undergoes chemical conversion by metabolic or chemical processes to yield a compound of Formula I or a salt thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

A co-crystal is a crystalline superstructure formed by combining an active pharmaceutical intermediate with an inert molecule and provides crystallinity to the combined form. Co-crystals are often made between a dicarboxylic acid such as fumaric acid, succinic acid etc. and a basic amine in different proportions depending on the nature of the co-crystal. (Remenar, J. F. et. al. J Am. Chem. Soc. 2003, 125, 8456).

All stereoisomers (for example, geometric isomers, optical isomers and the like) of the present compounds (including those of the salts, solvates, co-crystals and prodrugs of the compounds as well as the salts and solvates, co-crystals of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example the compounds of Formula Ia and Formula Ib). Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl and ¹²³I, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes. E.g., those labeled with positron-emitting isotopes like ¹¹C or ¹⁸F can be useful for application in Positron Emission Tomography (PET); and those labeled with gamma ray emitting isotopes like ¹²³I can be useful for application in Single photon emission computed tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Additionally, isotopic substitution at a site where epimerization occurs may slow or reduce the epimerization process and thereby retain the more active or efficacious form of the compound for a longer period of time. Isotopically-labeled compounds of Formula (I), in particular those containing isotopes with longer half lives (T1/2>1 day), can generally be prepared by following procedures analogous to those disclosed in the Schemes and/or in the Examples herein below, by substituting an appropriate isotopically labeled reagent for a non-isotopically labeled reagent.

As discussed above, the compounds of Formula I may be used to treat, ameliorate or prevent conditions associated with inhibiting the PAR-1 receptor. In addition to the conditions mentioned above, other conditions could include migraine, erectile dysfunction, rheumatoid arthritis, rheumatism, astrogliosis, a fibrotic disorder of the liver, kidney, lung or intestinal tract, systemic lupus erythematosus, multiple sclerosis, osteoporosis, renal disease, acute renal failure, chronic renal failure, renal vascular homeostasis, renal ischemia, bladder inflammation, diabetes, diabetic neuropathy, cerebral stroke, cerebral ischemia, nephritis, cancer, melanoma, renal cell carcinoma, neuropathy, malignant tumors, neurodegenerative and/or neurotoxic diseases, conditions or injuries, Alzheimer's disease, an inflammatory disease or condition, asthma, glaucoma, macular degeneration, psoriasis, endothelial dysfunction disorders of the liver, kidney or lung, inflammatory disorders of the lungs and gastrointestinal tract, respiratory tract disease or condition, radiation fibrosis, endothelial dysfunction, periodontal diseases or wounds, or a spinal cord injury, or a symptom or result thereof, viral infections, including infections from human respiratory syncytial virus (hRSV), human metapneumovirus (hMPV) and influenza virus type A, as well as other disorders in which thrombin and its receptor play a pathological role.

In another embodiment, compounds of the present invention might be expected to be useful in a method for treating, ameliorating or preventing radiation- and/or chemical-induced toxicity in non-malignant tissue in a patient comprising administering a therapeutically effective amount of at least one compound of Formula I or a pharmaceutically acceptable salt thereof. In particular, the radiation- and/or chemical-induced toxicity is one or more of intestinal fibrosis, pneumonitis, and mucositis. In one embodiment, the radiation- and/or chemical-induced toxicity is intestinal fibrosis. In another embodiment, the radiation- and/or chemical-induced toxicity is oral mucositis. In yet another embodiment, the radiation- and/or chemical-induced toxicity is intestinal mucositis, intestinal fibrosis, intestinal radiation syndrome, or pathophysiological manifestations of intestinal radiation exposure.

The present invention might also be expected to provide for methods for reducing structural radiation injury in a patient that will be exposed, is concurrently exposed, or was exposed to radiation and/or chemical toxicity, comprising administering a therapeutically effective amount of at least one compound of Formula I or a pharmaceutically acceptable salt thereof. The present invention might also be expected to provide for methods for reducing inflammation in a patient that will be exposed, is concurrently exposed, or was exposed to radiation and/or chemical toxicity, comprising administering a therapeutically effective amount of at least one compound of Formula I or a pharmaceutically acceptable salt thereof. The present invention might also be expected to provide for methods for adverse tissue remodeling in a patient that will be exposed, is concurrently exposed, or was exposed to radiation and/or chemical toxicity, comprising administering a therapeutically effective amount of at least one compound of Formula I or a pharmaceutically acceptable salt thereof. The present invention might also be expected to provide for methods for reducing fibroproliferative tissue effects in a patient that will be exposed, is concurrently exposed, or was exposed to radiation and/or chemical toxicity, comprising administering a therapeutically effective amount of at least one compound of Formula I or a pharmaceutically acceptable salt therof.

The present invention might also be expected to provide methods useful for treating a cell proliferative disorder in a patient by administering a therapeutically effective amount of at least one compound of Formula I or a pharmaceutically acceptable salt thereof. In one embodiment, the cell proliferative disorder is pancreatic cancer, glioma, ovarian cancer, colorectal and/or colon cancer, breast cancer, prostate cancer, thyroid cancer, lung cancer, melanoma, or stomach cancer. In one embodiment, the glioma is an anaplastic astrocytoma. In another embodiment, the glioma is a glioblastoma multiforme.

As used above, the term inflammatory disease or condition includes irritable bowel syndrome, Crohn's disease, nephritis or a radiation- or chemotherapy-induced proliferative or inflammatory disorder of the gastrointestinal tract, lung, urinary bladder, gastrointestinal tract or other organ. The term respiratory tract disease or condition includes reversible airway obstruction, asthma, chronic asthma, bronchitis or chronic airways disease. “Cancer” includes renal cell carcinoma or an angiogenesis related disorder. “Neurodegenerative disease” includes Parkinson's disease, amyotropic lateral sclerosis, Alzheimer's disease, Huntington's disease or Wilson's disease.

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically acceptable carrier. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

The amount and frequency of administration of the compound of this invention and/or their pharmaceutically acceptable salts will be regulated according to the judgment of the attending clinician considering such factors as age, condition and size of the patient as well as the severity of the symptoms being treated.

The quantity of active compound in a unit dose of preparation may be varied or adjusted from about 1 mg to about 150 mg, preferably from about 1 mg to about 75 mg, more preferably from about 1 mg to about 50 mg, according to the particular application.

The term “patient” includes animals, preferably mammals and especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk of said disease or medical condition.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. A “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. The terms “preventing” or “prevention” are used herein to refer to administering a compound before the onset of clinical symptoms.

The compounds of this invention may also be useful in combination (administered together or sequentially) with one or more therapeutic agents, such as, for example, another cardiovascular agent. Cardiovascular agents that could be used in combination with the compounds for Formula I or their pharmaceutically acceptable salts include drugs that have anti-thrombotic, anti-platelet aggregation, antiatherosclerotic, antirestenotic and/or anti-coagulant activity. Such drugs are useful in treating thrombosis-related diseases including thrombosis, atherosclerosis, restenosis, hypertension, angina pectoris, arrhythmia, heart failure, myocardial infarction, glomerulonephritis, thrombotic and thromboembolic stroke, peripheral vascular diseases, other cardiovascular diseases, cerebral ischemia, inflammatory disorders and cancer, as well as other disorders in which thrombin and its receptor play a pathological role. Suitable cardiovascular agents are selected from the group consisting of thromboxane A2 biosynthesis inhibitors such as aspirin; thromboxane antagonists such as seratrodast, picotamide and ramatroban; adenosine diphosphate (ADP) inhibitors such as clopidogrel; cyclooxygenase inhibitors such as aspirin, meloxicam, rofecoxib and celecoxib; angiotensin antagonists such as valsartan, telmisartan, candesartran, irbesartran, losartan and eprosartan; endothelin antagonists such as tezosentan; phosphodiesterase inhibitors such as milrinoone and enoximone; angiotensin converting enzyme (ACE) inhibitors such as captopril, enalapril, enaliprilat, spirapril, quinapril, perindopril, ramipril, fosinopril, trandolapril, lisinopril, moexipril and benazapril; neutral endopeptidase inhibitors such as candoxatril and ecadotril; valsartan/sacubitril (codenamed LCZ696); anticoagulants such as ximelagatran, fondaparin and enoxaparin; diuretics such as chlorothiazide, hydrochlorothiazide, ethacrynic acid, furosemide and amiloride; platelet aggregation inhibitors such as abciximab and eptifibatide; and GP IIb/IIIa antagonists.

Other possible combinations might include lipid lowering agents such as statins (e.g., simvastatin, lovastatin, pravastatin, atorvastatin, rosuvastatin, pitavastatin) and ezetimibe; niacin in immediate-release or controlled release forms or niacin in combination with a DP antagonist, such as laropiprant and/or with an HMG-CoA reductase inhibitor; niacin receptor agonists such as acipimox and acifran, as well as niacin receptor partial agonists; metabolic altering agents including insulin sensitizing agents and related compounds (e.g., muraglitazar, glipizide, stigliptin, metformin, rosiglitazone), PCSK9 inhibitors, e.g. antibodies—REGN727, AMG-145, RN316, RG7652; and small molecule inhibitors and CETP inhibitors, e.g., anacetrapib, evacetrapib, etc. Other possible combinations include AMPK agonists (e.g., ETC-1002); glucagon receptor antagonists; Lp-PLA2 inhibitors (e.g., darapladib) and anti-IL-1beta antibodies (canakinumab).

The dosage of the cardiovascular agent can be determined from published material, and may range from 1 to 1000 mg per dose.

An embodiment of this invention is combinations comprising an effective amount of a compound of Formula I or a pharmaceutically acceptable salt thereof and an ADP antagonist and/or cyclooxygenase inhibitor.

Non-limiting combinations comprise an effective amount of a compound according to Formula I or a pharmaceutically acceptable salt thereof and aspirin, ticagrelor, cangrelor, clopidogrel (either as a free base or as a pharmaceutically acceptable salt, such as its bisulfate salt), prasugrel, ticlopidine or fragmin.

Other therapeutic agents could include drugs that are known and used in the treatment of inflammation, rheumatism, asthma, glomerulonephritis, osteoporosis, neuropathy and/or malignant tumors, angiogenesis related disorders, cancer, disorders of the liver, kidney and lung, melanoma, renal cell carcinoma, renal disease, acute renal failure, chronic renal failure, renal vascular homeostasis, glomerulonephritis, chronic airways disease, bladder inflammation, neurodegenerative and/or neurotoxic diseases, conditions, or injuries, radiation fibrosis, endothelial dysfunction, periodontal diseases and wounds. Further examples of therapeutically effective agents which may be administered in combination with a compound of Formula I or a pharmaceutically acceptable salt thereof include resistance factors for tumor cells towards chemotherapy and proliferation inhibitors of smooth muscle cells, endothelial cells, fibroblasts, kidney cells, osteosarcoma cells, muscle cells, cancer cells and/or glial cells.

For treating and/or preventing radiation- and/or chemical-induced toxicity in non-malignant tissue, the present invention includes administering to a patient in need of such treatment an effective amount of a combination of one or more compounds of formula I and one or more radiation-response modifiers selected from the group consisting of Kepivance™ (palifermin), L-glutamine, teduglutide, sucralfate mouth rinses, iseganan, lactoferrin, mesna and trefoil factor.

For treating a cell proliferative disorder the present invention includes administering to a patient in need of such treatment an effective amount of a combination of one or more compounds of Formula I or a pharmaceutically acceptable salt thereof and another antineoplastic agent. In one embodiment, the other antineoplastic agent is temozolomide and the cell proliferative disorder is glioma. In another embodiment, the other antineoplastic agent is interferon and the cell proliferative disorder is melanoma. In one embodiment, the other antineoplastic agent is PEG-Intron (peginterferon alpha-2b) and the cell proliferative disorder is melanoma.

Pharmaceutical compositions comprising a therapeutically effective amount of a combination of at least one compound of Formula I or a pharmaceutically acceptable salt thereof and a radiation-response modifier in a pharmaceutically acceptable carrier are also provided.

Pharmaceutical compositions comprising a therapeutically effective amount of a combination of at least one compound of Formula I or a pharmaceutically acceptable salt thereof and an antineoplastic agent in a pharmaceutically acceptable carrier are also provided.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient.

Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), The Science and Practice of Pharmacy, 20^{th} Edition, (2000), Lippincott Williams & Wilkins, Baltimore, Md.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

In general, the compounds in the invention may be produced by a variety of processes known to those skilled in the art and by known processes analogous thereto. The invention disclosed herein is exemplified by the following preparations and examples which should not be construed to limit the scope of the disclosure. Alternative mechanistic pathways and analogous structures will be apparent to those skilled in the art. The practitioner is not limited to these methods.

Moreover, one skilled in the art would have resources such as Chemical Abstracts or Beilstein at his or her disposal to assist in preparing a specific compound.

One skilled in the art will recognize that one route will be optimized depending on the choice of appendage substituents. Additionally, one skilled in the art will recognize that in some cases the order of steps has to be controlled to avoid functional group incompatibility.

The prepared compounds may be analyzed for their composition and purity as well as characterized by standard analytical techniques such as, for example, elemental analysis, NMR, mass spectroscopy and IR spectra.

One skilled in the art will recognize that reagents and solvents actually used may be selected from several reagents and solvents well known in the art to be effective equivalents. Hence, when a specific solvent or reagent is mentioned, it is meant to be an illustrative example of the conditions desirable for that particular reaction scheme and in the preparations and examples described below.

Where NMR data are presented, 1H spectra were obtained, for example, on either a Varian Inova (400 or 500 mHz), Varian Mercury VX-400 (400 MHz), or Bruker-Biospin AV-500 (500 MHz), and are reported as ppm with number of protons and multiplicities indicated parenthetically. Where LC/MS data are presented, analyses was performed, for example, using an Agilent 1100 series or Applied Biosystems® API-100 mass spectrometer and C18 column, 5-95% CH₃ CN—H₂O (with 0.05% TFA) gradient. The observed parent ion is given.

Throughout the synthetic schemes, abbreviations are used with the following meaning unless otherwise indicated:

ACN or MeCN=acetonitrile;
Ac₂O=acetic anhydride;
AcOH=acetic acid;
Aq.=aqueous;
t-Butyl=tert-butyl;
t-BuOK=potassium tert-butoxide;
BrBn=benzyl bromide;
Cs₂CO₃=cesium carbonate;
DCM=dichloromethane;
DAST=diethylaminosulfur trifluoride;

### DIPA=Diisopropanolamine;

DMAP=4-dimethylamino pyridine;
DMP=Dess-Martin periodinane;
DMSO=dimethylsulfoxide;
Et=ethyl;
EtOH=ethanol;
EtOAc=ethyl acetate;
g=gas;
HEPES=4-(2-hydroxyethyl)-1-piperazineethanesulfonic acid;
HPLC=high pressure liquid chromatography;
IBX=2-iodoxybenzoic acid;
KOAc=Potassium acetate;
LCMS=liquid chromatorgraphy-mass spectrometry;
LDA=lithium diisopropyl amine;
LiHMDS=lithium bis(trimethylsilyl)amide;
Me=methyl;
MeCN=acetonitrile;
MeOH=methanol;
MeI=methyl iodide;
mmol=millimoles;
MsCl=mesylate chloride;
Na₂SO₄=sodium sulfate;
NH₄Cl=ammonium chloride;
NMP=N-methyl-2-pyrrolidone;
Pd(PPh₃)₄=Tetrakis(triphenylphosphine)palladium(0)
PDL=poly-D-lysine
Ph=phenyl;
Pet. Ether=petroleum ether
i-Pr=iso-propyl;
RT or rt=room temperature;

### SFC=Supercritical Fluid Chromatography;

TBAB=Tetra-n-butylammonium bromide;
TEA=triethanolamine;
TFA=trifluoroacetic acid;
THF=tetrahydrofuran; and
TLC=thin layer chromatography.

### EXAMPLES

### Intermediate Synthesis

Intermediate compounds of the present invention can be synthesized according to the schemes and procedures outlined below. Since the schemes are an illustration, the invention should not be construed as being limited by the chemical reactions and conditions expressed. The preparation of the various starting materials used in the schemes is well within the ordinary skill level of a practitioner of this art. Unless otherwise indicated, the definition for a variable is the same as that provided in Formula I.

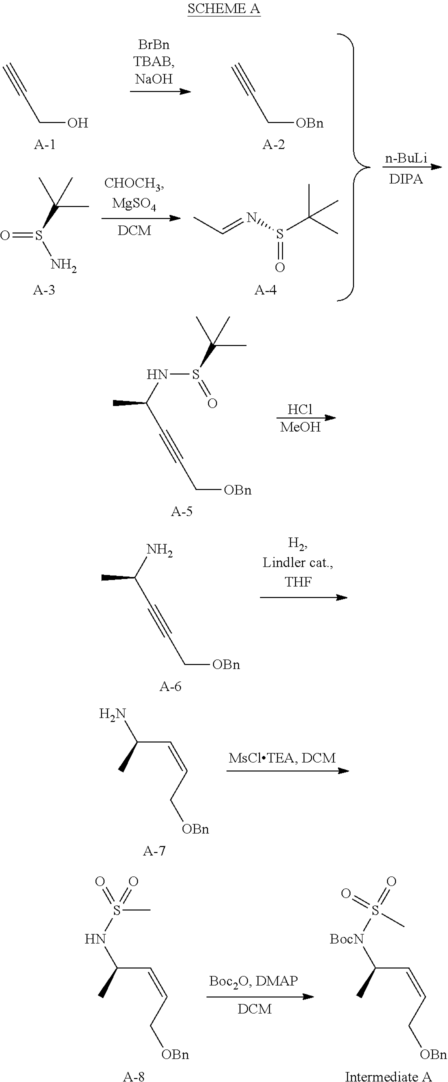

Intermediate A is prepared from commercially available alcohol (A-1) and sulfinamide (A-3) according to scheme A. Protection of alcohol (A-1) by benzyl bromide provides alkyne (A-2). Condensation of sulfinamide (A-3) with acetaldehyde affords imine (A-4). Anionic addition of terminal alkyne (A-2) to imine (A-4) provides adduct (A-5). Deprotection is achieved under the acidic conditions to provide amine (A-6), which is subsequently reduced under an atmosphere of H_{2(g)} in the presence of Lindlar's catalyst to yield cis-alkene (A-7). Alkene (A-7) is mesylated to afford sulfonamide (A-8) and subsequent Boc-protection provided intermediate A.

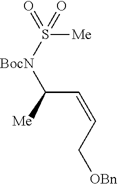 [SMILES: C[C@H](/C=C\COCc1ccccc1)N(C(=O)OC(C)(C)C)S(C)(=O)=O]

### Intermediate A

### (R,Z)-tert-butyl 5-(benzyloxy)pent-3-en-2-yl(methylsulfonyl)carbamate (Scheme A)

### Step 1: [(prop-2-yn-1-yloxy)methyl]benzene

(Bromomethyl)benzene (916 g, 5.09 mol, 95%), prop-2-yn-1-ol (300 g, 5.35 mol), TBAB (1725 g, 5.35 mol) and toluene (2.4 L) were added to a 10000-mL 4-necked round-bottom flask. The mixture was heated to 50° C. o this mixture was added a solution of sodium hydroxide (214.3 g) in water (500 mL) dropwise over a period of 1 hour. he resulting solution was stirred for an additional 3 h at 50° C. The reaction mixture was cooled to room temperature and the organic phase was separated, washed with brine (2×1500 mL), dried over anhydrous sodium sulfate and concentrated under vacuum. he crude product was purified by distillation under reduced pressure (10 mmHg) and the fraction was collected at 70° C. to yield the title compound.

### Step 2: (R)—N-[(1E)-ethylidene]-2-methylpropane-2-sulfinamide

Acetaldehyde (1018.2 g, 21.96 mol, 95%), (R)-2-methylpropane-2-sulfinamide (700 g, 5.78 mol), magnesium sulfate (3471.1 g, 28.36 mol) and dichloromethane (3500 mL) were added to a 10000-mL 4-necked round-bottom flask under an atmosphere of nitrogen. The resulting mixture was stirred overnight at room temperature. he solid was removed under filtration and the filtrate was concentrated under reduced pressure. This resulted in the title compound, which was carried forward without purification.

### Step 3: (R)—N-[(2R)-5-(benzyloxy)pent-3-yn-2-yl]-2-methylpropane-2-sulfinamide

A solution of diisopropylamine (539.6 g, 5.07 mol) in THF (4000 mL) was added to a 20000-mL 4-necked round-bottom flask under an atmosphere of nitrogen. This was followed by the addition of n-BuLi (1973 mL, 1.20 equiv) dropwise with stirring at −30° C. over 1 h. The mixture was stirred for 1 h at −30° C., before a solution of [(prop-2-yn-1-yloxy)methyl]benzene (600 g, 4.10 mol) in THF (1000 mL) was added dropwise with stirring at −78° C. over 30 min. The mixture was stirred for 30 min at −78° C., followed by addition of a solution of (R)—N-[(1E)-ethylidene]-2-methylpropane-2-sulfinamide (605 g, 4.11 mol) in THF (1000 mL) dropwise with stirring at −78° C. over 30 min. The resulting solution was warmed to room temperature and the reaction was quenched by the addition of saturated aqueous NH₄Cl (4 L). The resulting solution was extracted with ethyl acetate (2×2 L). The organic layers were combined, washed with brine (2 L), dried over anhydrous sodium sulfate and concentrated under vacuo. The residue was purified by silica gel chromatography (ethyl acetate/petroleum ether, 1/10) to yield the title compound.

### Step 4: (2R)-5-(benzyloxy)pent-3-yn-2-amine

(R)—N-[(2R)-5-(benzyloxy)pent-3-yn-2-yl]-2-methylpropane-2-sulfinamide (650 g, 2.10 mol), methanol (5000 mL) and HCl (370 mL) was added to a 10000-mL 4-necked round-bottom flask. The resulting solution was stirred for 1 h at room temperature. Volatiles were removed under reduced pressure and the residue was diluted with water (2 L) and EtOAc (2.5 L). The aqueous phase was adjusted to pH 10 with sodium carbonate, then extracted with ethyl acetate (2×2 L). The organic layers were combined, washed with brine (2 L), dried over anhydrous sodium sulfate and concentrated under vacuo to yield the title compound. LCMS (M+H)⁺ 190.

### Step 5: (R,Z)-5-(benzyloxy)pent-3-en-2-amine

Under H₂ atmosphere, a mixture of (R)-5-(benzyloxy)pent-3-yn-2-amine (18 g, 95.2 mmol), Lindlar's catalyst (1.8 g) and quinoline (2.8 mL, 23.8 mmol) in THF (200 mL) was stirred at room temperature for 2 h. After the reaction was completed, the mixture was filtered and concentrated under reduced pressure to afford crude compound. LCMS (M+H)⁺ 192.

### Step 6: (R,Z)—N-(5-(benzyloxy)pent-3-en-2-yl)methanesulfonamide

MsCl (9.6 mL, 124 mmol) was added dropwise to a of solution of (R,Z)-5-(benzyloxy)pent-3-en-2-amine (21 g, 95.2 mmol) and TEA (20 mL, 142.8 mmol) in 300 mL DCM at −20° C. The reaction mixture was stirred at −20° C. for 1 h. After the reaction was complete, the mixture was quenched with ice-cold water (10 mL), diluted with DCM (500 mL), washed with 3 N HCl (aq.) (200 mL×3), water (200 mL), and brine (200 mL), dried over Na₂SO₄ and concentrated under reduced pressure to afford crude compound. LCMS (M+Na)⁺ 292.

### Step 7: (R,Z)-tert-butyl-5-(benzyloxy)pent-3-en-2-yl)methylsulfonyl)carbamate

Boc₂O (41.6 g, 191 mmol) was added to a solution of (R,Z)—N-(5-(benzyloxy)pent-3-en-2-yl)methanesulfonamide (25.7 g, 95.2 mmol) and DMAP (0.58 g, 4.78 mmol) in DCM (300 mL) at room temperature. After 1 h, the reaction was concentrated under reduced pressure. The residues were purified by column chromatography on silica gel (ethyl acetate/pet. ether, 1/20) to afford the title compound. ¹H NMR (400 MHz, CDCl₃) δ 7.37-7.27 (m, 5H), 5.97-5.90 (m, 1H), 5.68-5.71 (m, 1H), 5.25-5.17 (m, 1H), 4.52 (s, 3H), 4.16-4.12 (m, 2H), 3.18 (s, 3H), 1.54 (s, 9H), 1.49 (d, 2H, J=6.8 Hz). LCMS (M+Na)⁺ 392.

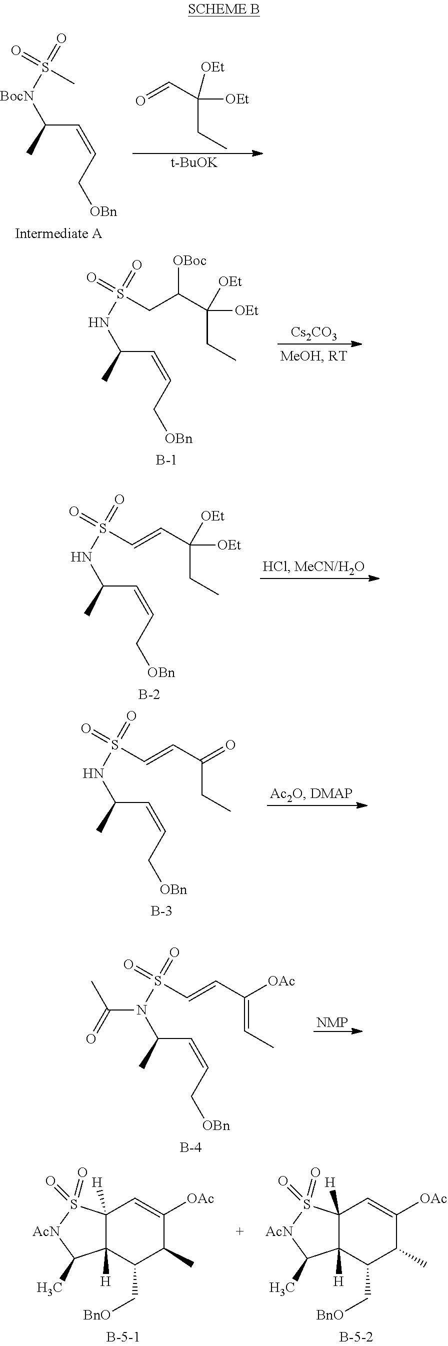

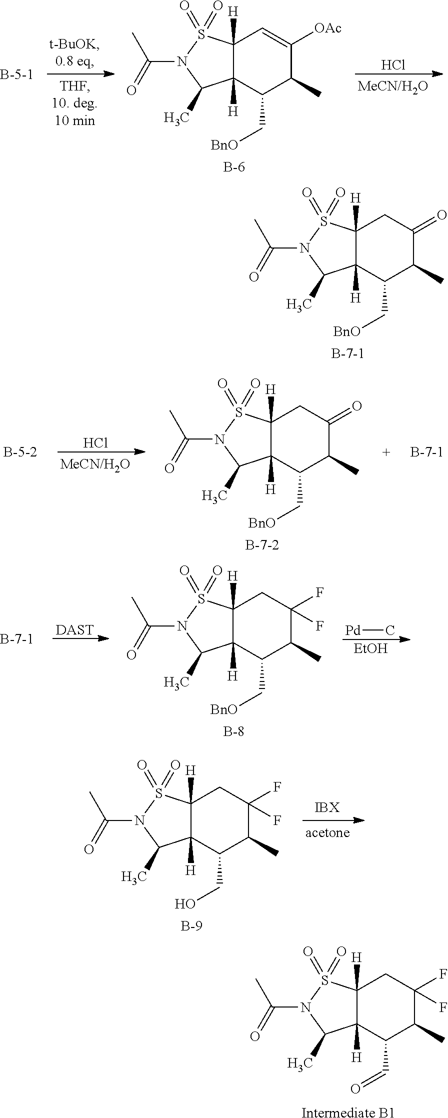

Condensation of methanesulfonamide (Intermediate A) with 2,2-diethoxybutanal (Babler, J. H. Syn. Commun. 1989, 19, 355-358) yielded a mixture of adduct (B-1) and eliminated adduct (B-2). Elimination of the carbonate from (B-1) under the action of Cs₂CO₃ provided acetal (B-2). Deprotection of the acetal by dilute HCl affords enone (B-3). Acetylation of (B-3) provides crude enol acetate (B-4), which is carried forward into an intramolecular Diels-Alder reaction that yield the exo and endo isomers (B-5-1 and B-5-2, respectively). The endo isomer (B-5-2) can be transformed to (B-7-1) by diluted HCl; the exo isomer (B-5-1) is treated with t-BuOK to afford a thermodynamically-favored isomer (B-6). Acid hydrolysis of (B-6) also provides ketone (B-7-1). Treatment of ketone (B-7-2) with DAST provides the corresponding des-fluoro compound (B-8). Deprotection of benzyl ether (B-8) under hydrogenation conditions affords alcohol (B-9) and subsequent oxidation by IBX affords intermediate B1.

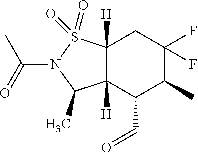 [SMILES: CC(=O)N1[C@H](C)[C@H]2[C@@H](C=O)[C@H](C)C(F)(F)C[C@H]2S1(=O)=O]

### Intermediate B1

### (3R,3aR,4S,5S,7aR)-2-acetyl-6,6-difluoro-3,5-dimethyloctahydrobenzo[d]isothiazole-4-carbaldehyde 1,1-dioxide

### Step 1: O-Boc-N—((R,Z)-5-(benzyloxy)pent-3-en-2-yl)-3,3-diethoxy-2-hydroxypentane-1-sulfonamide

A solution of t-BuOK (2.8 g, 25.0 mmol) in THF (28 mL) was added dropwise to a solution of (R,Z)-tert-butyl-5-(benzyloxy)pent-3-en-2-yl)methylsulfonyl)carbamate (4.2 g, 11.4 mmol) in 50 mL of THF at −78° C. The reaction mixture was stirred at −78° C. for 1 h, before a solution of 2,2-diethoxybutanal (2.2 g, 13.6 mmol) in THF (20 mL) was added dropwise. After the addition, the mixture was warmed to room temperature and was stirred overnight. The reaction was quenched with saturated aqueous NH₄Cl and extracted with EtOAc (200 mL×2). The combined organic layers were washed with brine, dried over anhydrous Na₂SO₄ and concentrated to afford crude compound.

### Step 2: (E)-N—((R,Z)-5-(benzyloxy)pent-3-en-2-yl)-3,3-diethoxypent-1-ene-1-sulfonamide

Crude O-Boc-N—((R,Z)-5-(benzyloxy)pent-3-en-2-yl)-3,3-diethoxy-2-hydroxypentane-1-sulfonamide (5.0 g) was dissolved in methanol (100 mL) and Cs₂CO₃ (7.4 g, 22.7 mmol) was added at room temperature. The mixture was stirred at room temperature overnight. Brine (300 mL) was added and the mixture was extracted with EtOAc (500 mL). The combined organic layer was dried over anhydrous Na₂SO₄, concentrated and purified by column chromatography on silica gel (ethyl acetate:hexanes, 1:8) to afford the title compound. LCMS (M+Na)⁺ 434.

### Step 3: (E)-N—((R,Z)-5-(benzyloxy)pent-3-en-2-yl)-3-oxopent-1-ene-1-sulfonamide

HCl (10 mL, 3 M) was added to a solution of (E)-N—((R,Z)-5-(benzyloxy)pent-3-en-2-yl)-3,3-diethoxypent-1-ene-1-sulfonamide (3.8 g, 9.2 mmol) in MeCN (20 mL) at room temperature. The reaction mixture was stirred for 2 h before brine (30 mL) was added. The mixture was extracted with EtOAc (30 mL), dried by anhydrous Na₂SO₄, and concentrated to afford the title compound, which was used immediately in the next step. LCMS (M+Na)⁺ 360.

### Step 4: (1E,3Z)-1-(N-acetyl-N—((R,Z)-5-(benzyloxy)pent-3-en-2-yl)sulfamoyl)penta-1,3-dien-3-yl acetate

(E)-N—((R,Z)-5-(benzyloxy)pent-3-en-2-yl)-3-oxopent-1-ene-1-sulfonamide (2.0 g, 5.9 mmol) was dissolved in acetic anhydride (20 mL) and DMAP (0.72 g, 5.9 mmol) was added at room temperature. The mixture was heated at 50° C. overnight and was cooled to room temperature before being poured into ice-cooled water (100 mL) with vigorous stirring for 10 min. The mixture was extracted with ethyl acetate (100 mL) and the organic layer was washed successively with aqueous saturated sodium bicarbonate (100 mL), brine (100 mL) and then concentrated to afford the title compound. LCMS (M+Na)⁺ 444.

### Step 5: (3R,3aR,4S,5S,7aR)-2-acetyl-4-((benzyloxy)methyl)-3,5-dimethyl-1,1-dioxido-2, 3,3a,4,5,7a-hexahydrobenzo[d]isothiazol-6-yl acetate and (3R,3aR,4S,5R,7aS)-2-acetyl-4-((benzyloxy)methyl)-3,5-dimethyl-1,1-dioxido-2,3,3a,4,5,7a-hexahydrobenzo[d]isothiazol-6-yl acetate

A solution of (1E,3Z)-1-(N-acetyl-N—((R,Z)-5-(benzyloxy)pent-3-en-2-yl) sulfamoyl)penta-1,3-dien-3-yl acetate (55 g, 102 mmol) in NMP (1 L) was heated at 140° C. for 8 h. The reaction mixture was poured onto water (4 L) and the aqueous layer was extracted with ethyl acetate (1 L). The separated organic layer was washed successively with water (1 L×2), brine (1 L), dried over anhydrous Na₂SO₄, concentrated and purified by column chromatography on silica gel (ethyl acetate/hexanes, 1/6) to afford a mixture of two isomers. Chiral resolution of the two aza-indane diastereomers was achieved by chiral preparative SFC (IC-H column, 80%/20% methanol/CO₂) to afford:

(3R,3aR,4S,5S,7aR)-2-acetyl-4-((benzyloxy)methyl)-3,5-dimethyl-1,1-dioxido-2,3,3a,4,5,7a-hexahydrobenzo[d]isothiazol-6-yl acetate (endo isomer, faster eluting). LCMS (M+H)⁺ 444. ¹H NMR (400 MHz, CDCl₃) δ 7.39-7.27 (m, 5H), 4.56-4.37 (m, 3H, J=11.6, 5.6 Hz), 3.69 (d, 1H, J=13.5 Hz), 3.55-3.39 (m, 2H), 2.58-2.49 (m, 1H), 2.42 (s, 3H), 2.40-2.30 (m, 1H), 2.26-2.19 (m, 1H), 2.17 (s, 3H), 1.41 (d, 3H, J=6.0 Hz), 1.23 (d, 3H, J=7.2 Hz) and

(3R,3 aR,4S,5R,7aS)-2-acetyl-4-((benzyloxy)methyl)-3,5-dimethyl-1,1-dioxido-2,3,3a,4,5,7a-hexahydrobenzo[d]isothiazol-6-yl acetate (exo isomer, slower eluting). LCMS (M+H)⁺ 444. ¹H NMR (400 MHz, CDCl₃) δ 7.49-7.28 (m, 5H), 5.54 (d, J=2.5 Hz, 1H), 4.67-4.44 (m, 3H), 4.14-4.02 (m, 1H), 3.67-3.51 (m, 2H), 2.92-2.79 (m, 1H), 2.60 (m, 2H), 2.37 (s, 3H), 2.17 (s, 3H), 1.34 (d, J=6.2 Hz, 3H), 1.07 (d, J=7.3 Hz, 3H).

### Step 6: (3R,3aR,4S,5S,7aS)-2-acetyl-4-((benzyloxy)methyl)-3,5-dimethyl-1,1-dioxido-2,3,3a,4,5,7a-hexahydrobenzo[d]isothiazol-6-yl acetate

(3R,3aR,4S,5S,7aR)-2-Acetyl-4-((benzyloxy)methyl)-3,5-dimethyl-1,1-dioxido-2,3,3a,4,5,7a-hexahydrobenzo[d]isothiazol-6-yl acetate (18 g, 42.7 mmol) was dissolved in THF (250 mL) and cooled to −50° C. A solution of potassium 2-methylpropan-2-olate (1.438 g, 12.81 mmol) in THF (20 mL) was added dropwise. The reaction mixture was stirred at −50° C. and the reaction was quenched with AcOH (1 mL). The reaction was diluted with brine (200 mL) and was extracted with EtOAc (300 mL). The separated organic layer was concentrated to afford crude compound, which was used directly in next step. LCMS (M+Na)⁺ 444.

### Step 7: (3R,3aR,4S,5S,7aR)-2-acetyl-4-((benzyloxy)methyl)-3,5-dimethylhexahydrobenzo[d]isothiazol-6(2H)-one 1,1-dioxide

Hydrogen chloride (90 mL, 4M in dioxanes, 360 mmol) was added to a mixture of (3R,3aR,4S,5R,7aS)-2-acetyl-4-((benzyloxy)methyl)-3,5-dimethyl-1,1-dioxido-2,3,3a,4,5,7a-hexahydrobenzo[d]isothiazol-6-yl acetate (6.0 g, 14.2 mmol) in MeCN (200 mL) at 5° C. The mixture was stirred at 0-10° C. for 4 days. The mixture was poured into brine (300 mL), and the aqueous layer was extracted with EtOAc (300 mL). The separated organic layer was washed successively with saturated aqueous sodium bicarbonate (200 mL), then brine (300 mL) before being concentrated. The residue was purified by silica gel chromatography (0-20% EtOAc/hexane) to afford the title compound. ¹H NMR (400 MHz, CDCl₃) δ 7.33 (m, 5H), 4.76-4.64 (m, 1H), 4.49 (dd, J=39.2, 11.6 Hz, 2H), 3.68-3.51 (m, 3H), 3.00-2.91 (m, 1H), 2.90-2.81 (m, 1H), 2.69-2.58 (m, 1H), 2.53-2.43 (m, 1H), 2.40 (s, 3H), 2.20-2.08 (m, 1H), 1.48 (d, J=5.9 Hz, 3H), 1.03 (d, J=6.5 Hz, 3H). LCMS (M+H)⁺ 380.

### Step 8: (3R,3aR,4S,5S,7aR)-2-acetyl-4-((benzyloxy)methyl)-3,5-dimethylhexahydrobenzo[d]isothiazol-6(2H)-one 1,1-dioxide

Hydrogen chloride (150 mL, 4M, 600 mmol) was added to a solution of (3R,3aR,4S,5S,7aS)-2-acetyl-4-((benzyloxy)methyl)-3,5-dimethyl-1,1-dioxido-2,3,3a,4,5,7a-hexahydrobenzo[d]isothiazol-6-yl acetate (18 g, 83% pure, 35.4 mmol) in MeCN (450 mL) at 5° C. The mixture was stirred at 5-10° C. for 4 days. The mixture was poured into brine (500 mL), extracted with EtOAc (1 L) and the organics were then washed with brine (3×500 mL) before concentration. The title compound was used directly in the next step without purification. LCMS (M+H)⁺ 380.

### Step 9: 1-((3R,3aR,4S,5S,7aR)-4-((benzyloxy)methyl)-6,6-difluoro-3,5-dimethyl-1,1-dioxidohexahydrobenzo[d]isothiazol-2(3H)-y)ethanone

(3R,3aR,4S,5S,7aR)-2-acetyl-4-((benzyloxy)methyl)-3,5 dimethylhexahydrobenzo[d]isothiazol-6(2H)-one 1,1-dioxide (21 g, 57% pure, 31.5 mmol) was dissolved in DCM (200 mL) and DAST (12.5 mL, 95 mmol) was added at 0° C. The reaction mixture was stirred at 0-15° C. for 6 h. The mixture was quenched with ice (50 g), neutralized with aqueous saturated NaHCO₃(400 mL). The separated organic layer was washed with brine (300 mL), concentrated and purified by column chromatography on silica gel (0-20% EtOAc/hexanes) to afford the title compound. LCMS (M+H)⁺ 402.

### Step 10: 1-((3R,3aR,4S,5S,7aR)-6,6-difluoro-4-(hydroxymethyl)-3,5-dimethyl-1,1-dioxidohexa hydrobenzo[d]isothiazol-2(3H)-yl)ethanone

1-((3R,3aR,4S,5S,7aR)-4-((benzyloxy)methyl)-6,6-difluoro-3,5-dimethyl-1,1-dioxidohexahydrobenzo[d]isothiazol-2(3H)-yl)ethanone (7.5 g, 18.7 mmol) was dissolved in methanol (100 mL) and Pd-C(3.6 g, 10% wt) was added. The mixture was placed under H_{2(g)} (1 atm) overnight at room temperature. The mixture was filtered and concentrated to afford the title compound. ¹H NMR (400 MHz, CDCl₃) δ 4.50-4.39 (m, 1H), 3.91 (d, J=10.9 Hz, 1H), 3.78 (dd, J=11.0, 6.7 Hz, 1H), 3.52-3.41 (m, 1H), 2.88-2.78 (m, 1H), 2.70-2.58 (m 1H), 2.39 (s, 3H), 2.13-1.95 (m, 2H), 1.85-1.64 (m, 2H), 1.51 (d, J=5.8 Hz, 3H), 1.12 (d, J=5.9 Hz, 3H). LCMS (M+H)⁺ 312.

### Step 11: (3R,3aR,4S,5S,7aR)-2-acetyl-6,6-difluoro-3,5-dimethyloctahydrobenzo[d]isothiazole-4-carbaldehyde 1,1-dioxide

1-((3R,3aR,4S,5S,7aR)-6,6-difluoro-4-(hydroxymethyl)-3,5-dimethyl-1,1-dioxidohexahydrobenzo[d]isothiazol-2(3H)-yl)ethanone (5.0 g, 16.1 mmol) was dissolved in acetone (100 mL) and IBX (7.0 g, 25.0 mmol) was added at room temperature. The reaction mixture was stirred at 40° C. for 24 h. The mixture was filtered, concentrated and purified by column chromatography on silica gel (EtOAc/hexane, 1/5) to afford the title compound. ¹H NMR (400 MHz, CDCl₃) δ 9.83 (t, J=1.8 Hz, 1H), 4.36-4.15 (m, 1H), 3.69-3.49 (m, 1H), 3.09-2.91 (m, 2H), 2.76-2.62 (m, 1H), 2.49-2.29 (m, 4H), 1.89-1.67 (m, 1H), 1.30 (d, J=5.9 Hz, 3H), 1.11 (d, J=6.5 Hz, 3H). LCMS (M+H)⁺ 310.

The following example in Table B was prepared according to Scheme B using the procedure outlined for the synthesis of Intermediate B1 from (3R,3aR,4S,5R,7aR)-2-acetyl-4-((benzyloxy)methyl)-3,5-dimethylhexahydrobenzo[d]isothiazol-6(2H)-one 1,1-dioxide:

TABLE B

| | | | LLCMS |
|---|---|---|---|
| Intermediate | Structure | IUPAC Name | (M + H)⁺ |
| B2 | 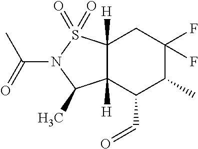 [SMILES: CC(=O)N1[C@H](C)[C@H]2[C@@H](C=O)[C@@H](C)C(F)(F)C[C@H]2S1(=O)=O] | (3R,3aR,4S,5R,7aR)-2-acetyl-6,6-difluoro- 3,5-dimethyloctahydrobenzo[d]isothiazole- 4-carbaldehyde 1,1-dioxide | 310 |

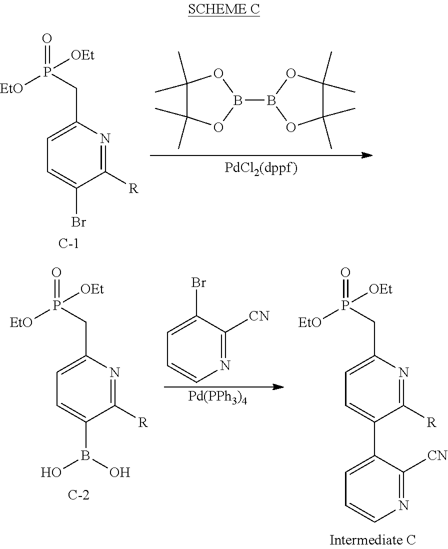

Intermediate C is prepared conveniently from known bromides (C-1) by Miyaura borylation (C-2) and Suzuki reaction.

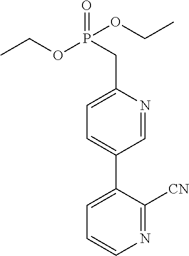 [SMILES: CCOP(=O)(Cc1ccc(-c2cccnc2C#N)cn1)OCC]

### Intermediate C

### Diethyl (2′-cyano-3,3′-bipyridin-6-yl)methylphosphonate (Scheme C)

### Step 1: 6-((diethoxyphosphoryl)methyl)pyridin-3-ylboronic acid

A flask was charged with diethyl (5-bromopyridin-2-yl)methylphosphonate (12.3 g, 40 mmol), bis(pinacolato)diboron (12.2 g, 48 mmol), 1,1′-bis(diphenylphosphino)ferrocene-palladium(II) dichloride (600 mg, 0.82 mmol), KOAc (11.8 g, 120 mmol) and dioxane (200 mL). The mixture was purged with argon for 5 minutes and was heated at 100° C. for 1 h. The solids were filtered off and the filtrate was concentrated to afford the title compound, which was used in the next step without purification. LCMS (M+H)⁺ 274.

### Step 2: diethyl (2′-cyano-3,3′-bipyridin-6-yl)methylphosphonate

A flask was charged with 6-((diethoxyphosphoryl)methyl)pyridin-3-ylboronic acid (20 g), 3-bromopicolinonitrile (8.1 g, 44 mmol), Pd(PPh₃)₄ (1.4 g, 1.2 mmol), NaHCO₃ (10 g, 120 mmol), dioxane (200 mL) and water (60 mL). The mixture was purged with argon for 5 minutes and then was heated to 100° C. for 16 h. The reaction was diluted with water (200 mL) and EtOAc (200 mL) the organic later was washed with brine (300 mL), dried over anhydrous sodium sulfate, and concentrated. The crude product was purified by chromatography on silica gel (0-1/25 MeOH in DCM) to afford the title compound. ¹H NMR (400 MHz, DMSO) δ 8.86-8.74 (m, 2H), 8.22 (dd, J=8.0, 1.3 Hz, 1H), 8.10 (dd, J=8.1, 2.3 Hz, 1H), 7.87 (dd, J=8.0, 4.7 Hz, 1H), 7.56 (dd, J=8.1, 2.1 Hz, 1H), 4.10-3.98 (m, 4H), 3.56 (d, J=22.0 Hz, 2H), 1.21 (t, J=7.0 Hz, 6H). LCMS (M+H)⁺ 332.

The following example in Table C was prepared according to Scheme C using the procedure outlined for the synthesis of Intermediate Cl from known or commercially available materials.

TABLE C

| | | | LLCMS |
|---|---|---|---|
| Intermediate | Structure | IUPAC Name | (M + H)⁺ |
| C2 | 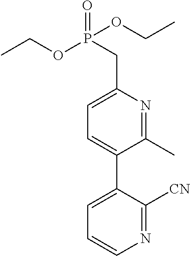 [SMILES: CCOP(=O)(Cc1ccc(-c2cccnc2C#N)c(C)n1)OCC] | diethyl (2′-cyano-2-methyl-3,3′-bipyridin-6- yl)methylphosphonate | 346 |

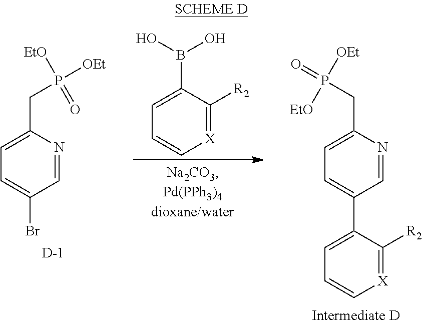

Intermediate D is prepared from known phosphonate ester (D-1, Chelliah, M. V., et. al. J. Med. Chem. 2007, 50, 5147-5160) and commercially available boronic acids or esters via Suzuki reaction.

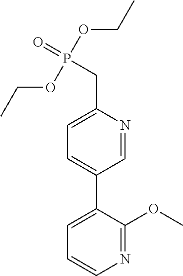 [SMILES: CCOP(=O)(Cc1ccc(-c2cccnc2OC)cn1)OCC]

### Intermediate D1

### Diethyl (2′-methoxy-3,3′-bipyridin-6-yl)methylphosphonate

### Step 1: diethyl ((2′-methoxy-[3,3′-bipyridin]-6-yl)methyl) phosphonate

A flask was charged with diethyl ((5-bromopyridin-2-yl)methyl)phosphonate (4.5 g, 14.6 mmol), (2-methoxypyridin-3-yl)boronic acid (3.5 g, 22.9 mmol), Pd(Ph₃P)₄ (1 g, 0.87 mmol), Na₂CO₃ (3 g, 0.03 mmol) in dioxane (50 mL) and water (12 mL). The mixture was purged with argon for 5 minutes and was stirred at 90° C. for 5 h. The reaction was diluted with water (100 mL) and EtOAc (150 mL). The aqueous layer was back-extracted with EtOAc (100×3) and the combined organic layers were washed with brine (100 mL), dried over Na₂SO₄, filtered, concentrated and purified by column chromatography on silica gel (EtOAc/PE=1.5/1 and MeOH/DCM=20/1) to afford the title compound. LCMS (M+H)⁺ 337.

The following example in Table D was prepared according to scheme D using the procedure outlined for the synthesis of Intermediate D1 from known or commercially available materials.

TABLE D

| | | | LLCMS |
|---|---|---|---|
| Intermediate | Structure | IUPAC Name | (M + H)⁺ |
| D2 | 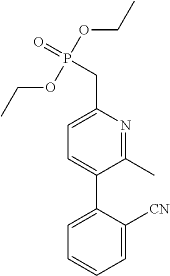 [SMILES: CCOP(=O)(Cc1ccc(-c2ccccc2C#N)c(C)n1)OCC] | diethyl (5-(2-cyanophenyl)-6-methylpyridin-2- yl)methylphosphonate | 345 |

### General Synthetic Schemes

Representative compounds of the present invention can be synthesized according to the general schemes outlined below as well as the representative examples that follow. Since the schemes are an illustration, the invention should not be construed as being limited by the chemical reactions and conditions expressed. The preparation of the various starting materials used in the schemes is well within the skill of a person versed in the art.

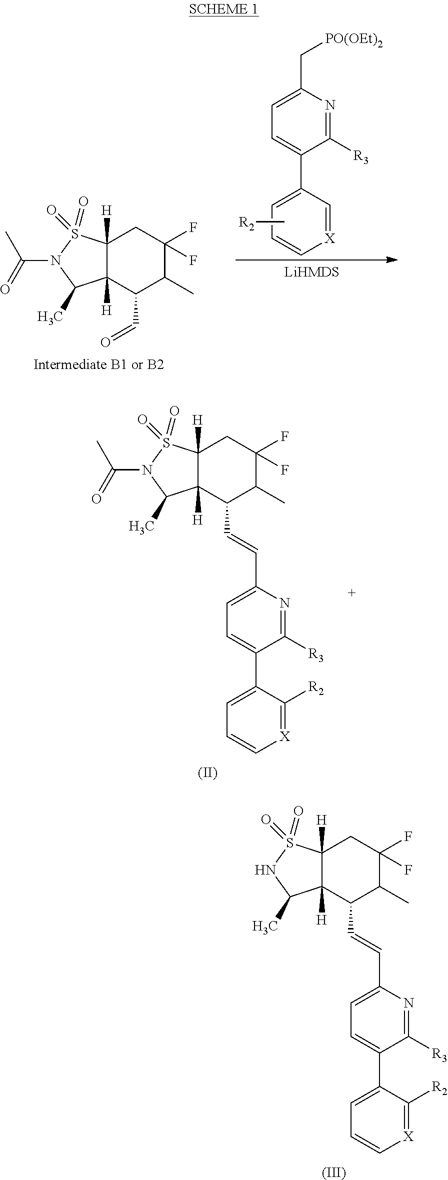

Compounds of Formula (II) and (III) are prepared by a Hoerner-Wadsworth-Emmons olefination reaction from prepared aldehydes (Intermediate B) with prepared or known phosphonate esters.

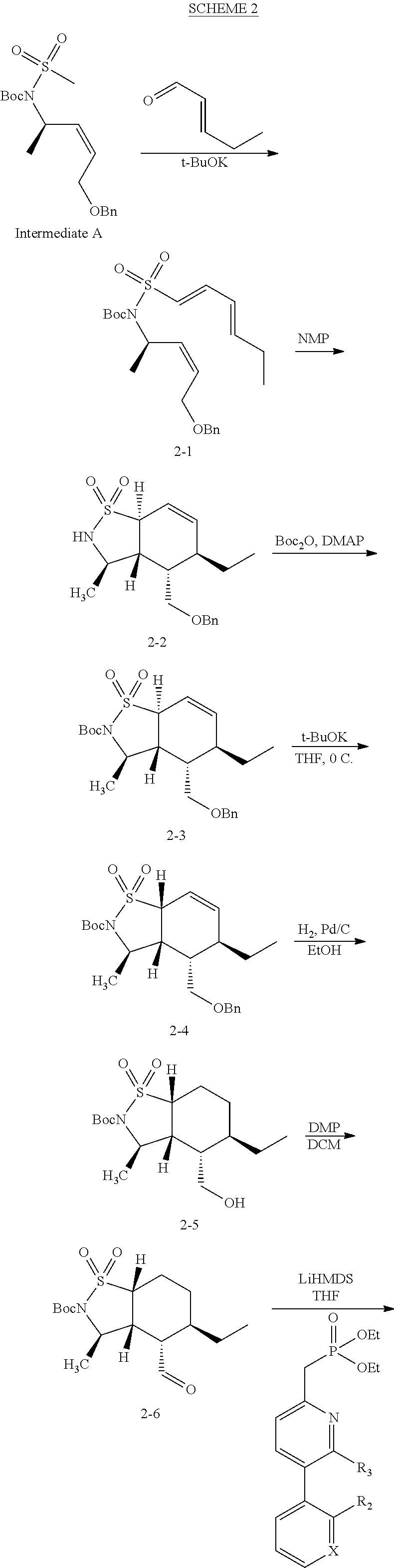

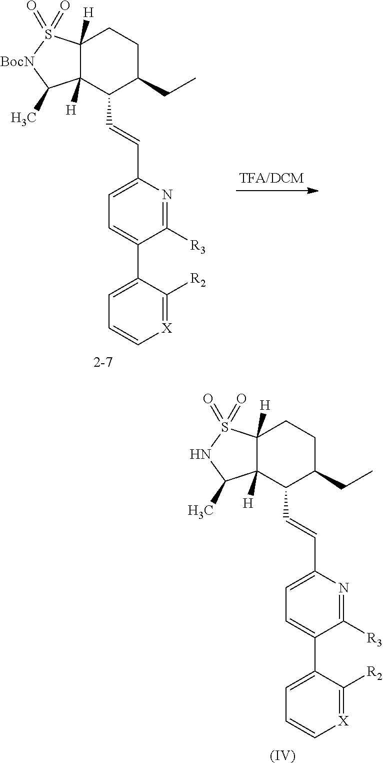

Compounds of Formula (IV) are synthesized beginning from the condensation of Intermediate A and (E)-pent-2-enal to form the precursor (2-1) for an intramolecular Diels-Alder reaction. Cyclization provides sulfonamide (2-2), which is the exo-isomer and the major product.

Subsequent Boc-protection yields (2-3), which is then transformed to a thermodynamically-favored configuration under the action of base to yield the cis-fused isomer (2-4). Hydrogenation reveals alcohol (2-5), which is then oxidized by DMP to afford aldehyde (2-6). Olefination using a Hoerner-Wadsworth-Emmons reaction with known or prepared phosphonate esters provides alkene (2-7). Deprotection by TFA provides compound of formula (IV).

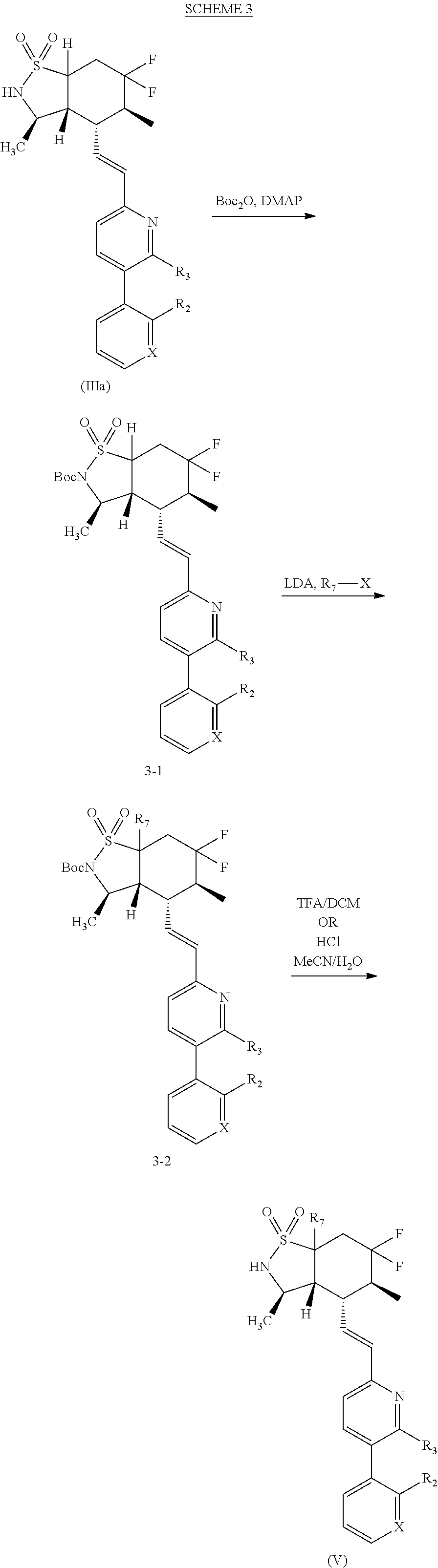

Compounds of Formula (V) are prepared according to Scheme 3 beginning with the Boc-protection of compound (Ma) to provide (3-1). Alkylation by commericially available electrophiles yields adduct (3-2). Subsequent Boc-deprotection and/or hydrolysis provides compounds of the Formula (V).

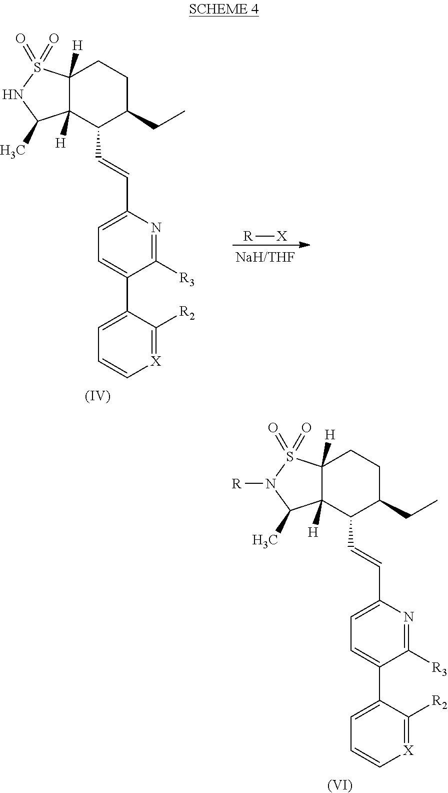

Compounds of Formula (VI) are synthesized from compounds of Formula (IV) under alkylation conditions with commerically available electrophiles.

The following schemes and examples are provided so that the invention will be more fully appreciated and understood. Starting materials are made using known procedures or as illustrated below.

### Example 1

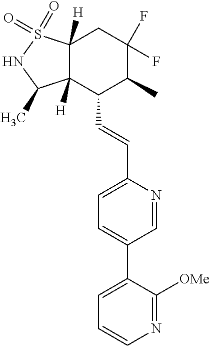 [SMILES: COc1ncccc1-c1ccc(/C=C/[C@@H]2[C@H]3[C@@H](CC(F)(F)[C@H]2C)S(=O)(=O)N[C@@H]3C)nc1]

### (3R,3aR,4S,5S,7aR)-6,6-difluoro-4-((E)-2-(2′-methoxy-[3,3′-bipyridin]-6-yl)vinyl)-3,5-dimethyloctahydrobenzo[d]isothiazole 1,1-dioxide (Scheme 1)

LiHMDS (1 M in THF, 9.5 mL, 9.50 mmol) was added to a solution of diethyl ((2′-methoxy-[3,3′-bipyridin]-6-yl)methyl)phosphonate (3.26 g, 9.70 mmol) in THF (30 mL) at 0° C. After addition was complete, the mixture was stirred at 0° C. for 30 min. A solution of (3R,3aR,4S,5S,7aR)-2-acetyl-6,6-difluoro-3,5-dimethyloctahydrobenzo[d]isothiazole-4-carbaldehyde 1,1-dioxide (1.0 g, 3.23 mmol) in THF (10 mL) was added dropwise to the reaction. The mixture was stirred at room temperature for 3 h, and was then quenched with saturated aqueous NH₄Cl (100 mL). The aqueous layer was extracted with EtOAc (50 mL×3) and the combined organic layers were washed with brine (100 mL), dried over anhydrous Na₂SO₄, filtered, concentrated and purified by column chromatography on silica gel (20-34% EtOAc/hexanes) to afford the title compound. ¹H NMR (400 MHz, CDCl₃) δ 8.75 (d, J=2.1 Hz, 1H), 8.21 (dd, J=5.0, 1.8 Hz, 1H), 7.89 (dd, J=8.1, 2.1 Hz, 1H), 7.64 (dd, J=7.3, 1.8 Hz, 1H), 7.25 (d, J=7.4 Hz, 1H), 7.02 (dd, J=7.3, 5.0 Hz, 1H), 6.67-6.56 (m, 2H), 4.64 (d, J=4.7 Hz, 1H), 3.99 (s, 3H), 3.83-3.74 (m, 1H), 3.52-3.40 (m, 1H), 2.88-2.74 (m, 2H), 2.74-2.62 (m, J=14.6, 5.7 Hz, 1H), 2.13-1.92 (m, 2H), 1.43-1.36 (m, 3H), 1.08 (d, J=6.6 Hz, 3H). LCMS (M+H)⁺ 450. PAR-1 FLIPR IC₅₀=5.1 nM

This reaction also afforded a minor N-acetylated product, which can be hydrolyzed to: 1-((3R,3aR,4S,5S,7aR)-6,6-difluoro-4-((E)-2-(2′-methoxy-[3,3′-bipyridin]-6-yl)vinyl)-3,5-dimethyl-1,1-dioxidohexahydrobenzo[d]isothiazol-2(3H)-yl)ethanone. ¹H NMR (400 MHz, CDCl₃) δ 8.76 (d, J=2.1 Hz, 1H), 8.21 (dd, J=5.0, 1.8 Hz, 1H), 7.90 (dd, J=8.1, 2.1 Hz, 1H), 7.64 (dd, J=7.3, 1.9 Hz, 1H), 7.32-7.27 (m, 1H), 7.02 (dd, J=7.3, 5.0 Hz, 1H), 6.71-6.54 (m, J=15.4, 12.2 Hz, 2H), 4.49-4.40 (m, J=11.7, 5.8 Hz, 1H), 3.99 (s, 3H), 3.64-3.53 (m, 1H), 2.86-2.77 (m, 1H), 2.76-2.63 (m, 2H), 2.42 (d, J=6.0 Hz, 3H), 2.12-1.97 (m, J=18.1, 12.9, 5.5 Hz, 1H), 1.91-1.71 (m, 1H), 1.41 (d, J=7.7 Hz, 3H), 1.08 (d, J=6.6 Hz, 3H). LCMS (M+H)⁺ 492.

The following examples in Table 1 were prepared according to Scheme 1 using the procedure outlined in the synthesis of Example 1 from prepared or commercially available materials.

TABLE 1

| | | | LCMS | PAR-1 |
|---|---|---|---|---|
| Ex. | Structure | IUPAC Name | [M + H]+ | FLIPR IC₅₀ (nM) |
| 2 | 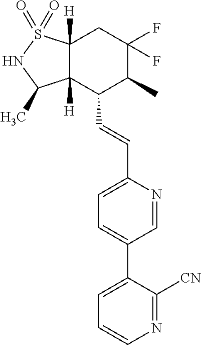 [SMILES: C[C@H]1NS(=O)(=O)[C@@H]2CC(F)(F)[C@@H](C)[C@H](/C=C/c3ccc(-c4cccnc4C#N)cn3)[C@@H]21] | (3R,3aR,4S,5S,7aR)-6,6-difluoro-4-((E)-2-(2′- cyano-[3,3′-bipyridin]-6-yl)vinyl)-3,5- dimethyloctahydrobenzo[d]isothiazole 1,1- dioxide | 445 | 55.8 |
| 3 | 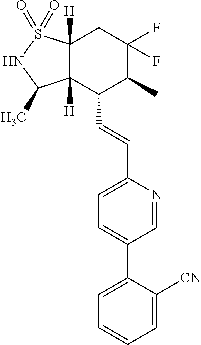 [SMILES: C[C@H]1NS(=O)(=O)[C@@H]2CC(F)(F)[C@@H](C)[C@H](/C=C/c3ccc(-c4ccccc4C#N)cn3)[C@@H]21] | (3R,3aR,4S,5S,7aR)-4-((E)-2-(5-(2- cyanophenyl)pyridin-2-yl)vinyl)-6,6-difluoro- 3,5-dimethyloctahydrobenzo[d]isothiazole- 1,1-dioxide | 444 | 11.9 |
| 4 | 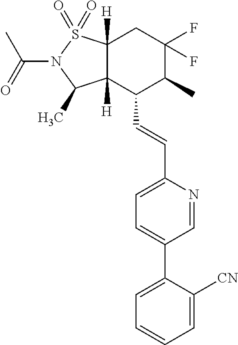 [SMILES: CC(=O)N1[C@H](C)[C@H]2[C@@H](/C=C/c3ccc(-c4ccccc4C#N)cn3)[C@H](C)C(F)(F)C[C@H]2S1(=O)=O] | 1-((3R,3aR,4S,5S,7aR)-6,6-difluoro-4-((E)-2- (5-(2-cyanophenyl)pyridin-2-yl)vinyl)-3,5- dimethyl-1,1- dioxidohexahydrobenzo[d]isothiazol-2(3H)- yl)ethanone | 486 | 483 |
| 5 | 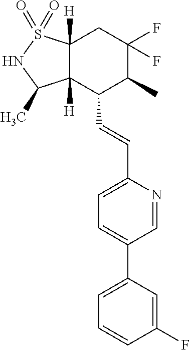 [SMILES: C[C@H]1NS(=O)(=O)[C@@H]2CC(F)(F)[C@@H](C)[C@H](/C=C/c3ccc(-c4cccc(F)c4)cn3)[C@@H]21] | (3R,3aR,4S,5S,7aR)-6,6-difluoro-4-((E)-2-(5- (3-fluorophenyl)pyridin-2-yl)vinyl)-3,5- dimethyloctahydrobenzo[d]isothiazole 1,1- dioxide | 437 | 19 |
| 6 | 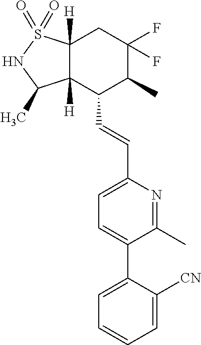 [SMILES: Cc1nc(/C=C/[C@@H]2[C@H]3[C@@H](CC(F)(F)[C@H]2C)S(=O)(=O)N[C@@H]3C)ccc1-c1ccccc1C#N] | (3R,3aR,4S,5S,7aR)-4-((E)-2-(5-(2- cyanophenyl)pyridin-2-yl)-6-methylvinyl)- 6,6-difluoro-3,5- dimethyloctahydrobenzo[d]isothiazole-1,1- dioxide | 458 | 66.7 |
| 7 | 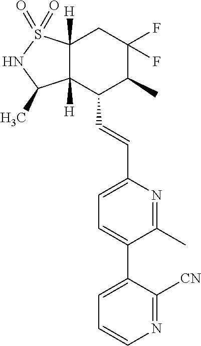 [SMILES: Cc1nc(/C=C/[C@@H]2[C@H]3[C@@H](CC(F)(F)[C@H]2C)S(=O)(=O)N[C@@H]3C)ccc1-c1cccnc1C#N] | (3R,3aR,4S,5S,7aR)-4-((E)-2-(5-(2- cyanophenyl)pyridin-2-yl)-6-methylvinyl)- 6,6-difluoro-3,5- dimethyloctahydrobenzo[d]isothiazole-1,1- dioxide | 459 | 41 |
| 8 | 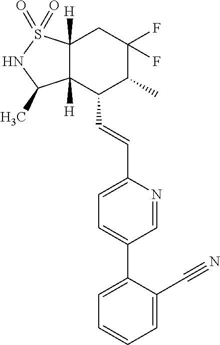 [SMILES: C[C@@H]1[C@H](/C=C/c2ccc(-c3ccccc3C#N)cn2)[C@H]2[C@@H](CC1(F)F)S(=O)(=O)N[C@@H]2C] | (3R,3aR,4S,5R,7aR)-4-((E)-2-(5-(2- cyanophenyl)pyridin-2-yl)vinyl)-6,6-difluoro- 3,5-dimethyloctahydrobenzo[d]isothiazole- 1,1-dioxide | 444 | 178 |
| 9 | 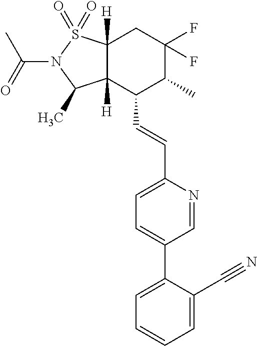 [SMILES: CC(=O)N1[C@H](C)[C@H]2[C@@H](/C=C/c3ccc(-c4ccccc4C#N)cn3)[C@@H](C)C(F)(F)C[C@H]2S1(=O)=O] | 1-((3R,3aR,4S,5R,7aR)-6,6-difluoro-4-((E)-2- (5-(2-cyanophenyl)pyridin-2-yl)vinyl)-3,5- dimethyl-1,1- dioxidohexahydrobenzo[d]isothiazol-2(3H)- yl)ethanone | 486 | 564 |

### Example 10

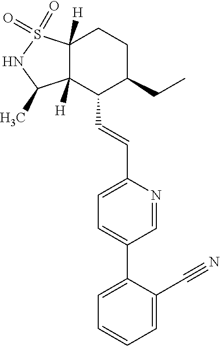 [SMILES: CC[C@@H]1CC[C@@H]2[C@H]([C@H]1/C=C/c1ccc(-c3ccccc3C#N)cn1)[C@@H](C)NS2(=O)=O]

### 2-(6-((E)-2-((3R,3aR,4R,5R,7aR)-5-ethyl-3-methyl-1,1-dioxidooctahydrobenzo[d]isothiazol-4-yl)vinyl)pyridin-3-yl)benzonitrile (Scheme 2)

### Step 1: (1E,3E)-N—((R,Z)-5-(benzyloxy)pent-3-en-2-yl)hexa-1,3-diene-1-sulfonamide

A solution of t-BuOK (5.46 g, 48.8 mmol) in THF (100 mL) was added dropwise to a solution of (R,Z)-tert-butyl 5-(benzyloxy)pent-3-en-2-yl(methylsulfonyl) carbamate (9.0 g, 24.4 mmol) in THF (100 mL) at −78° C. After addition was complete, the mixture was stirred at −78° C. for 1 h. A solution of (E)-pent-2-enal (3.07 g, 36.6 mmol) in THF (10 mL) was added dropwise at the same temperature. After addition, the reaction mixture was warmed to room temperature and was stirred overnight. The reaction was quenched with saturated aqueous NH₄Cl (400 mL) and extracted with EtOAc (500 mL×2). The combined organic layers were washed with brine (200 mL), dried over anhydrous Na₂SO₄, and concentrated. The residues were purified by column chromatography on silica gel (EtOAc/petroleum ether=1/6) to afford the title compound. LCMS (M+Na)⁺ 358.2.

### Step 2: (3R,3aR,4S,5R,7aS)-4-((benzyloxy)methyl)-5-ethyl-3-methyl-2,3,3a,4,5,7a-hexahydro benzo[d]isothiazole 1,1-dioxide

A mixture of (1E,3E)-N—((R,Z)-5-(benzyloxy)pent-3-en-2-yl)hexa-1,3-diene-1-sulfonamide (6.3 g, 18.8 mmol) in NMP (350 mL) was stirred at 180° C. for 11 h. The reaction mixture was diluted in EtOAc (2 L), washed with water (250 mL×4) and brine (250 mL), dried over anhydrous Na₂SO₄ and concentrated under vacuo to afford the title compound. The crude material was carried forward into the next step without purification. LCMS (M+H)⁺ 336.

### Step 3: (3R,3aR,4S,5R,7aS)-tert-butyl-((benzyloxy)methyl)-5-ethyl-3-methyl-3,3a,4,5-tetrahydro benzo[d]isothiazole-2(7aH)-carboxylate 1,1-dioxide

Boc₂O (8.2 g, 37.6 mmol) was added to a solution of (3R,3aR,4S,5R,7aS)-4-((benzyloxy)methyl)-5-ethyl-3-methyl-2,3,3a,4,5,7a-hexahydrobenzo[d]isothiazole 1,1-dioxide (6.3 g, 18.8 mmol) and DMAP (0.12 g, 0.94 mmol) in DCM (100 mL) at room temperature. The reaction mixture was stirred for 0.5 h and then concentrated and purified by column chromatography on silica gel (EtOAc/petroleum ether=1/30) to afford the title compound. LCMS (2M+Na)⁺ 893.

### Step 4: (3R,3aR,4S,5R,7aR)-tert-butyl-((benzyloxy)methyl)-5-ethyl-3-methyl-3,3a,4,5-tetrahydro benzo[d]isothiazole-2(7aH)-carboxylate 1,1-dioxide

A solution of t-BuOK (0.237 g, 2.115 mmol) in THF (5 mL) was added dropwise to a solution of (3R,3 aR,4S,5R,7aS)-tert-butyl-((benzyloxy)methyl)-5-ethyl-3-methyl-3,3a,4,5-tetrahydrobenzo[d]isothiazole-2(7aH)-carboxylate 1,1-dioxide (2.3 g, 5.29 mmol) in THF (25 mL) at 0° C. After stirring for 15 min at 0° C., the reaction was quenched with saturated aqueous NH₄Cl (200 mL) and extracted with EtOAc (200 mL×2). The combined organic layers were washed with brine (100 mL), dried over anhydrous Na₂SO₄, and concentrated and purified by column chromatography on silica gel (1/20-1/10 EtOAc in pet. ether) to afford the title compound. ¹H NMR (400 MHz, CDCl₃) δ 7.38-7.28 (m, 5H), 6.00-5.95 (m, 1H), 5.88-5.82 (m, 1H), 4.53-4.43 (m, 2H), 4.15-4.00 (m, 1H), 3.78-3.76 (m, 1H), 3.66 (dd, 1H, J=3.6 Hz, 9.6 Hz), 3.43 (t, 1H, J=9.2 Hz), 2.97-2.82 (m, 1H), 2.22-2.12 (m, 1H), 2.04-1.98 (m, 1H), 1.66-1.58 (m, 1H), 1.51 (s, 9H), 1.41 (d, 2H, J=6.0 Hz), 1.38-1.28 (m, 1H), 0.88 (t, 3H, J=7.6 Hz). LCMS (2M+Na)⁺ 893.

### Step 5: (3R,3aR,4S,5R,7aR)-tert-butyl 5-ethyl-4-(hydroxymethyl)-3-methylhexahydrobenzo[d]isothiazole-2(3H)-carboxylate 1,1-dioxide

Pd—C(1.74 g, 10 wt %) was added to a solution of (3R,3aR,4S,5R,7aR)-tert-butyl-((benzyloxy)methyl)-5-ethyl-3-methyl-3,3a,4,5-tetrahydrobenzo[d]isothiazole-2(7aH)-carboxylate 1,1-dioxide (1.74 g, 4 mmol) in methanol (100 mL). The mixture was placed under 1 atm of hydrogen at 25° C. for 40 h. The reaction mixture was filtered and concentrated to afford the title compound. The crude material was carried forward to the next step without purification. LCMS (2M+Na)⁺ 717.

### Step 6: (3R,3aR,4S,5R,7aR)-tert-butyl 5-ethyl-4-formyl-3-methylhexahydrobenzo[d]isothiazole-2(3H)-carboxylate 1,1-dioxide

DMP (3.1 g, 7.32 mmol) was added to a solution of (3R,3aR,4S,5R,7aR)-tert-butyl 5-ethyl-4-(hydroxymethyl)-3-methylhexahydrobenzo[d]isothiazole-2(3H)-carboxylate 1,1-dioxide (1.27 g, 3.66 mmol) in DCM (50 mL) at room temperature. After stirring at room temperature for 2 h, the mixture was quenched by aqueous Na₂SO₃ (100 mL, 5 w/wt %) and neutralized with aqueous NaHCO₃ (100 mL, 10%). The product was extracted with DCM (100 mL×2) and was washed with aqueous NaHCO₃ (100 mL, 10%), then brine (100 mL), dried over anhydrous Na₂SO₄, and concentrated to afford the title compound. LCMS (M+Na)⁺ 368.

### Step 7: (3R,3aR,4R,5R,7aR)-tert-butyl 4-((E)-2-(5-(2-cyanophenyl)pyridin-2-yl) vinyl)-5-ethyl-3-methylhexahydrobenzo[d]isothiazole-2(3H)-carboxylate1,1-dioxide

Lithium bis(trimethylsilyl)amide (5.8 mL, 5.8 mmol, 1 M) in THF was added dropwise to a solution of diethyl (5-(2-cyanophenyl)pyridin-2-yl)methylphosphonate (1.91 g, 5.8 mmol) in THF (10 mL) at −20° C. After the addition was complete, the reaction mixture was stirred at 0° C. for 30 minutes. The reaction was warmed to room temperature and titanium(IV) isopropoxide (1.75 mL, 5.8 mmol) was added and the mixture was stirred for 5 min. A solution of (3R,3aR,4S,5R,7aR)-tert-butyl 5-ethyl-4-formyl-3-methylhexahydrobenzo[d]isothiazole-2(3H)-carboxylate 1,1-dioxide (0.8 g, 2.32 mmol) in THF (10 mL) was addded at room temperature, and the reaction was stirred for 0.5 h. The reaction was quenched with aqueous saturated potassium sodium tartrate (50 mL) and the aqueous was extracted with EtOAc (200 mL×2). The combined organic layers were dried over anhydrous Na₂SO₄, and purified by column chromatography on silica gel (1/10-1/5 EtOAc in pet. ether) to afford the title compound. ¹H NMR (400 MHz, CDCl₃) δ 8.71 (d, 1H, J=2.4 Hz), 7.98-7.90 (m, 1H), 7.81 (d, 1H, J=7.6 Hz), 7.71 (t, 1H, J=7.2 Hz), 7.55-7.49 (m, 2H), 6.80-6.60 (m, 2H), 4.23-4.13 (m, 1H), 3.25-3.18 (m, 1H), 3.60-3.52 (m, 1H), 2.50-2.40 (m, 1H), 2.30-2.20 (m, 1H), 2.10-2.05 (m, 1H), 1.70-1.55 (m, 2H), 1.53 (s, 9H), 1.41 (d, 3H, J=5.6 Hz), 1.37-1.31 (m, 1H), 1.15-0.95 (m, 2H), 0.88 (t, 3H, J=7.2 Hz). LCMS (M+H)⁺ 522. PAR-1 FLIPR IC₅₀=286 nM

### Step 8: 2-(6-((E)-2-((3R,3aR,4R,5R,7aR)-5-ethyl-3-methyl-1,1-dioxidooctahydrobenzo[d]iso thiazol-4-yl)vinyl)pyridin-3-yl)benzonitrile

A mixture of (3R,3aR,4R,5R,7aR)-tert-butyl 4-((E)-2-(5-(2-cyanophenyl)pyridin-2-yl) vinyl)-5-ethyl-3-methylhexahydrobenzo[d]isothiazole-2(3H)-carboxylate 1,1-dioxide (0.18 g, 0.345 mmol) in TFA (3 mL) and DCM (3 mL) was stirred at room temperature for 2 h. The reaction mixture was concentrated, dissolved in MeOH (1 mL), neutralized by ammonia aqueous (17 wt %, 0.1 mL) and purified by HPLC (MeCN/water+0.08% NH₄HCO₃ modifier) to afford the title compound. ¹H NMR (400 MHz, CDCl₃) δ 8.73 (d, 1H, J=2.0 Hz), 7.91 (d, 1H, J=7.6 Hz), 7.81 (d, 1H, J=7.6 Hz), 7.70 (t, 1H, J=8.0 Hz), 7.54-7.49 (m, 2H), 7.32 (d, 1H, J=8.0 Hz), 6.80-6.55 (m, 2H), 4.39 (d, 1H, J=5.6 Hz), 3.82-3.70 (m, 1H), 3.23-3.13 (m, 1H), 2.80-2.72 (m, 1H), 2.53-2.43 (m, 1H), 2.30-2.20 (m, 1H), 2.10-2.02 (m, 1H), 1.70-1.41 (m, 3H), 1.36 (d, 3H, J=6.0 Hz), 1.15-0.95 (m, 2H), 0.88 (t, 3H, J=7.6 Hz). LCMS (M+H)⁺ 422. PAR-1 FLIPR IC₅₀=0.97 nM

### Example 11

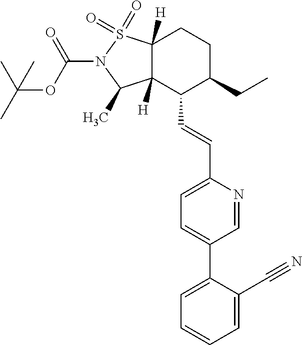 [SMILES: CC[C@@H]1CC[C@@H]2[C@H]([C@H]1/C=C/c1ccc(-c3ccccc3C#N)cn1)[C@@H](C)N(C(=O)OC(C)(C)C)S2(=O)=O]

### (3R,3aR,4R,5R,7aR)-tert-butyl 4-((E)-2-(5-(2-cyanophenyl)pyridin-2-yl) vinyl)-5-ethyl-3-methylhexahydrobenzo[d]isothiazole-2(3H)-carboxylate1,1-dioxide

Lithium bis(trimethylsilyl)amide (5.8 mL, 5.8 mmol, 1 M) in THF was added dropwise to a solution of diethyl (5-(2-cyanophenyl)pyridin-2-yl)methylphosphonate (1.91 g, 5.8 mmol) in THF (10 mL) at −20° C. After the addition was complete, the reaction mixture was stirred at 0° C. for 30 minutes. The reaction was warmed to room temperature and titanium(IV) isopropoxide (1.75 mL, 5.8 mmol) was added and the mixture was stirred for 5 min. A solution of (3R,3aR,4S,5R,7aR)-tert-butyl 5-ethyl-4-formyl-3-methylhexahydrobenzo[d]isothiazole-2(3H)-carboxylate 1,1-dioxide (0.8 g, 2.32 mmol) in THF (10 mL) was addded at room temperature, and the reaction was stirred for 0.5 h. The reaction was quenched with aqueous saturated potassium sodium tartrate (50 mL) and the aqueous was extracted with EtOAc (200 mL×2). The combined organic layers were dried over anhydrous Na₂SO₄, and purified by column chromatography on silica gel (1/10-1/5 EtOAc in pet. ether) to afford the title compound. ¹H NMR (400 MHz, CDCl₃) δ 8.71 (d, 1H, J=2.4 Hz), 7.98-7.90 (m, 1H), 7.81 (d, 1H, J=7.6 Hz), 7.71 (t, 1H, J=7.2 Hz), 7.55-7.49 (m, 2H), 6.80-6.60 (m, 2H), 4.23-4.13 (m, 1H), 3.25-3.18 (m, 1H), 3.60-3.52 (m, 1H), 2.50-2.40 (m, 1H), 2.30-2.20 (m, 1H), 2.10-2.05 (m, 1H), 1.70-1.55 (m, 2H), 1.53 (s, 9H), 1.41 (d, 3H, J=5.6 Hz), 1.37-1.31 (m, 1H), 1.15-0.95 (m, 2H), 0.88 (t, 3H, J=7.2 Hz). LCMS (M+H)⁺ 522. PAR-1 FLIPR IC₅₀=286 nM

### Example 12

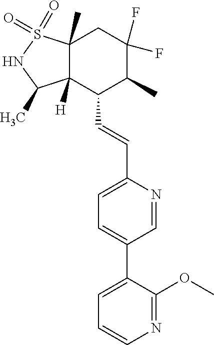 [SMILES: COc1ncccc1-c1ccc(/C=C/[C@@H]2[C@@H]3[C@@H](C)NS(=O)(=O)[C@]3(C)CC(F)(F)[C@H]2C)nc1]

### (3R,3aR,4S,5S,7aR)-6,6-difluoro-4-((E)-2-(2′-methoxy-[3,3′-bipyridin]-6-yl)vinyl)-3,5,7a-trimethyloctahydrobenzo[d]isothiazole 1,1-dioxide (Scheme 3)

### Step 1: (3R,3aR,4S,5S,7aR)-tert-butyl-6,6-difluoro-4-((E)-2-(2′-methoxy-[3,3′-bipyridin]-6-yl)vinyl)-3,5-dimethylhexahydrobenzo[d]isothiazole-2(3H)-carboxylate 1,1-dioxide (Example 12)

Boc₂O (0.851 mL, 3.67 mmol) was added to a solution of (3R,3aR,4S,5S,7aR)-6,6-difluoro-4-((E)-2-(2′-methoxy-[3,3′-bipyridin]-6-yl)vinyl)-3,5-dimethyloctahydrobenzo[d]isothiazole 1,1-dioxide (800 mg, 1.78 mmol) and DMAP (11 mg, 0.090 mmol) in DCM (12 mL). The mixture was stirred at 20° C. for 10 min and then was concentrated. The residue was purified by column chromatography on silica gel (EtOAc/pet. ether=1/4) to afford the title compound. ¹H NMR (400 MHz, CDCl₃) δ 8.77 (s, 1H), 8.22 (dd, J=5.0, 1.7 Hz, 1H), 7.90 (s, 1H), 7.64 (dd, J=7.3, 1.8 Hz, 1H), 7.26 (s, 1H), 7.05-6.98 (m, J=7.3, 5.0 Hz, 1H), 6.75-6.55 (m, 2H), 4.23-4.15 (m, J=11.8, 5.9 Hz, 1H), 3.99 (s, 3H), 3.55-3.43 (m, 1H), 2.86-2.75 (m, 1H), 2.74-2.64 (m, J=5.5 Hz, 2H), 2.18-2.05 (m, J=19.3 Hz, 1H), 2.02-1.82 (m, 1H), 1.54 (s, 9H), 1.45 (d, J=5.8 Hz, 3H), 1.08 (d, J=6.6 Hz, 3H). LCMS (M+H)⁺ 550. PAR-1 FLIPR IC₅₀=278 nM

### Step 2: (3R,3aR,4S,5S,7aR)-tert-butyl 6,6-difluoro-4-((E)-2-(2′-methoxy-[3,3′-bipyridin]-6-yl)vinyl)-3,5,7a-trimethylhexahydrobenzo[d]isothiazole-2(3H)-carboxylate 1,1-dioxide

A microwave tube was charged with (3R,3aR,4S,5S,7aR)-tert-butyl 6,6-difluoro-4-((E)-2-(2′-methoxy-[3,3′-bipyridin]-6-yl)vinyl)-3,5-dimethylhexahydrobenzo[d]isothiazole-2(3H)-carboxylate 1,1-dioxide (120 mg, 0.218 mmol) and THF (2 mL). The tube was purged with argon and then sealed and cooled to −78° C. LDA (0.22 mL, 2 M in THF, 0.440 mmol) was added dropwise over 5 min, and the reaction was stirred at −78° C. for 40 min. Iodomethane (90 mg, 0.634 mmol) was added, and the mixture was stirred for another 30 min at −78° C. The reaction mixture was quenched with saturated aqueous NH₄Cl (5 mL) and then partioned with EtOAc. The organic layer was washed with brine (10 mL), dried over anhydrous Na₂SO₄, filtered and concentrated to afford the title compound. LCMS (M+H)⁺ 564.

### Step 3: (3R,3aR,4S,5S,7aR)-6,6-difluoro-4-((E)-2-(2′-methoxy-[3,3′-bipyridin]-6-yl)vinyl)-3,5,7a-trimethyloctahydrobenzo[d]isothiazole 1,1-dioxide

A solution of (3R,3aR,4S,5S,7aR)-tert-butyl 6,6-difluoro-4-((E)-2-(2′-methoxy-[3,3′-bipyridin]-6-yl)vinyl)-3,5,7a-trimethylhexahydrobenzo[d]isothiazole-2(3H)-carboxylate 1,1-dioxide (150 mg, 0.266 mmol) in TFA (0.5 mL) and DCM (5 mL) was stirred at 20° C. for 2 h. The reaction was neutralized with saturated aqueous NaHCO₃ (10 mL) and the aqueous phase was extracted with DCM (5 mL×3). The combined organic layers were washed with saturated brine (10 mL), dried over anhydrous Na₂SO₄, filtered and concentrated to afford the title compound. ¹H NMR (400 MHz, CDCl₃) δ 8.78 (s, 1H), 8.23 (s, 1H), 7.98 (s, 1H), 7.65 (d, J=6.6 Hz, 1H), 7.28 (s, 1H), 7.04 (d, J=4.5 Hz, 1H), 6.67 (d, J=13.5 Hz, 2H), 4.50 (s, 1H), 4.00 (d, J=2.8 Hz, 3H), 3.74 (s, 1H), 2.75 (s, 1H), 2.47-1.99 (m, 4H), 1.59 (s, 3H), 1.55 (s, 1H), 1.40-1.32 (m, 3H), 1.09 (d, J=6.5 Hz, 3H). LCMS (M+H)⁺ 464. PAR-1 FLIPR IC₅₀=4.2 nM

The following Examples in Table 2 were prepared according to Scheme 3 using the procedure outlined for the synthesis of Example 12 from prepared or commercially available materials.

TABLE 2

| | | | LCMS | PAR-1 |
|---|---|---|---|---|
| Ex. | Structure | IUPAC Name | [M + H]+ | FLIPR IC₅₀ (nM) |
| 13 | 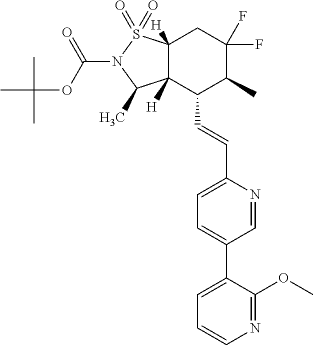 [SMILES: COc1ncccc1-c1ccc(/C=C/[C@@H]2[C@@H]3[C@@H](C)N(C(=O)OC(C)(C)C)S(=O)(=O)[C@@H]3CC(F)(F)[C@H]2C)nc1] | (3R,3aR,4S,5S,7aR)-tert-butyl 6,6- difluoro-4-((E)-2-(2′-methoxy-[3,3′- bipyridin]-6-yl)vinyl)-3,5- dimethylhexahydrobenzo[d]isothiazole- 2(3H)-carboxylate 1,1-dioxide | 550 | 278 |
| 14 | 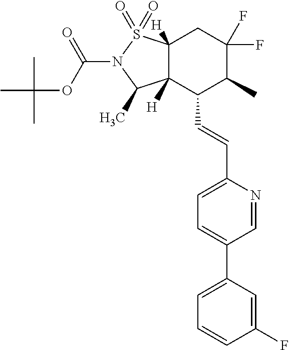 [SMILES: C[C@@H]1[C@H]2[C@@H](/C=C/c3ccc(-c4cccc(F)c4)cn3)[C@H](C)C(F)(F)C[C@H]2S(=O)(=O)N1C(=O)OC(C)(C)C] | (3R,3aR,4S,5S,7aR)-tert-butyl 6,6- difluoro-4-((E)-2-(5-(3- fluorophenyl)pyridin-2-yl)vinyl)-3,5- dimethylhexahydrobenzo[d]isothiazole- 2(3H)-carboxylate 1,1-dioxide | 537 | 383 |
| 15 | 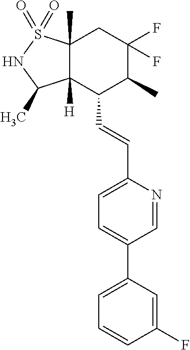 [SMILES: C[C@H]1NS(=O)(=O)[C@]2(C)CC(F)(F)[C@@H](C)[C@H](/C=C/c3ccc(-c4cccc(F)c4)cn3)[C@H]12] | (3R,3aR,4S,5S,7aR)-6,6-difluoro-4-((E)- 2-(5-(3-fluorophenyl)pyridin-2- yl)vinyl)-3,5,7a- trimethyloctahydrobenzo[d]isothiazole 1,1-dioxide | 451 | 55.1 |
| 16 | 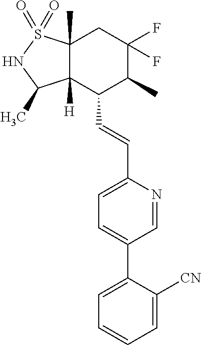 [SMILES: C[C@H]1NS(=O)(=O)[C@]2(C)CC(F)(F)[C@@H](C)[C@H](/C=C/c3ccc(-c4ccccc4C#N)cn3)[C@H]12] | (3R,3aR,4S,5S,7aR)-6,6-difluoro-4-((E)- 2-(5-(2-cyanophenyl)pyridin-2- yl)vinyl)-3,5,7a- trimethyloctahydrobenzo[d]isothiazole 1,1-dioxide | 458 | 11.8 |
| 17 | 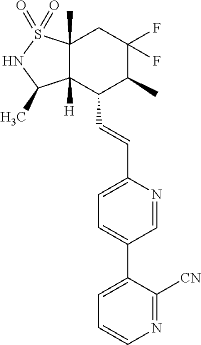 [SMILES: C[C@H]1NS(=O)(=O)[C@]2(C)CC(F)(F)[C@@H](C)[C@H](/C=C/c3ccc(-c4cccnc4C#N)cn3)[C@H]12] | (3R,3aR,4S,5S,7aR)-6,6-difluoro-4-((E)- 2-(2′-cyano-[3,3′-bipyridin]-6-yl)vinyl)- 3,5,7a- trimethyloctahydrobenzo[d]isothiazole 1,1-dioxide | 459 | 12 |

### Example 18

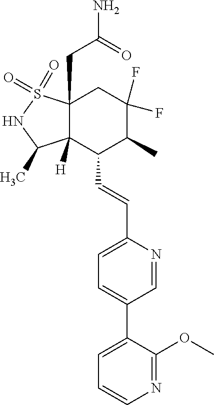 [SMILES: COc1ncccc1-c1ccc(/C=C/[C@@H]2[C@@H]3[C@@H](C)NS(=O)(=O)[C@]3(CC(N)=O)CC(F)(F)[C@H]2C)nc1]

### 2-((3R,3aR,4S,5S,7aR)-6,6-difluoro-4-((E)-2-(2′-methoxy-[3,3′-bipyridin]-6-yl)vinyl)-3,5-dimethyl-1,1-dioxidooctahydrobenzo[d]isothiazol-7a-yl)acetamide (Scheme 3)

### Step 1: (3R,3 aR,4S,5S,7aR)-tert-butyl-7a-(2-amino-2-oxoethyl)-6,6-difluoro-4-((E)-2-(2′-methoxy-[3,3′-bipyridin]-6-yl)vinyl)-3,5-dimethylhexahydrobenzo[d]isothiazole-2(3H)-carboxylate 1,1-dioxide

A microwave tube was charged with (3R,3aR,4S,5S,7aR)-tert-butyl 6,6-difluoro-4-((E)-2-(2′-methoxy-[3,3′-bipyridin]-6-yl)vinyl)-3,5-dimethylhexahydrobenzo[d]isothiazole-2(3H)-carboxylate 1,1-dioxide (260 mg, 0.473 mmol) and THF (5 mL). The mixture was purged with argon and then sealed and cooled to −78° C. LDA (0.6 mL, 2 M in THF, 1.20 mmol) was added and the reaction was stirred for 40 min at −78° C. 2-Bromoacetamide (100 mg, 0.725 mmol) in THF (1.5 mL) was added to the reaction at −78° C. and the mixture was stirred for an additional 30 min. The reaction was quenched with saturated aqueous NH₄Cl (6 mL) at −78° C. and was diluted with EtOAc (20 mL) and water (20 mL). The organic layer was separated, the aqueous phase was extracted with EtOAc (20 mL×2). The combined organic layers were washed with brine (10 mL), dried over anhydrous Na₂SO₄, filtered, concentrated, and purified by column chromatography on silica gel (20-100% EtOAc/pet. ether) to afford the title compound. ¹H NMR (400 MHz, MeOD) δ 8.71 (s, 1H), 8.19 (d, J=4.9 Hz, 1H), 8.01 (d, J=8.2 Hz, 1H), 7.80 (d, J=7.3 Hz, 1H), 7.57 (d, J=8.2 Hz, 1H), 7.14-7.04 (m, 1H), 6.84-6.62 (m, 2H), 4.37-4.27 (m, 1H), 3.98 (d, J=1.4 Hz, 3H), 3.11-2.84 (m, 4H), 2.47-2.28 (m, 2H), 2.13 (ddd, J=29.2, 14.8, 11.7 Hz, 1H), 1.54 (d, J=0.5 Hz, 9H), 1.37 (d, J=8.0 Hz, 3H), 1.08 (d, J=6.5 Hz, 3H).

LCMS (M+1)′ 607. PAR-1 FLIPR IC₅₀=110 nM

### Step 2: 2-((3R,3aR,4S,5S,7aR)-6,6-difluoro-4-((E)-2-(2′-methoxy-[3,3′-bipyridin]-6-yl)vinyl)-3,5-dimethyl-1,1-dioxidooctahydrobenzo[d]isothiazol-7a-yl)acetamide

A solution of (3R,3aR,4S,5S,7aR)-tert-butyl 7a-(2-amino-2-oxoethyl)-6,6-difluoro-4-((E)-2-(2′-methoxy-[3,3′-bipyridin]-6-yl)vinyl)-3,5-dimethylhexahydrobenzo[d]isothiazole-2(3H)-carboxylate 1,1-dioxide (140 mg, 0.231 mmol) in TFA (0.6 mL) and DCM (6 mL) was stirred at 20° C. for 2 h. The acid was neutralized with saturated aqueous NaHCO₃ (10 mL) and the aqueous layer was extracted with DCM (10 mL×3). The combined organic layers were washed with brine (10 mL), dried over anhydrous Na₂SO₄, filtered and concentrated to afford the title compound. ¹H NMR (400 MHz, MeOD) δ 8.59 (d, J=2.1 Hz, 1H), 8.09 (dd, J=4.9, 1.6 Hz, 1H), 7.90 (dd, J=8.1, 2.1 Hz, 1H), 7.70 (d, J=7.4 Hz, 1H), 7.45 (d, J=8.2 Hz, 1H), 7.00 (dd, J=7.3, 5.0 Hz, 1H), 6.67-6.50 (m, 2H), 3.88 (s, 3H), 3.68-3.56 (m, 1H), 2.93-2.64 (m, 4H), 2.15 (ddd, J=20.2, 17.2, 3.6 Hz, 3H), 1.16 (d, J=6.7, 3H), 0.97 (d, J=6.6 Hz, 3H). LCMS (M+H)⁺ 507. PAR-1 FLIPR IC₅₀=36.8 nM

### Example 19

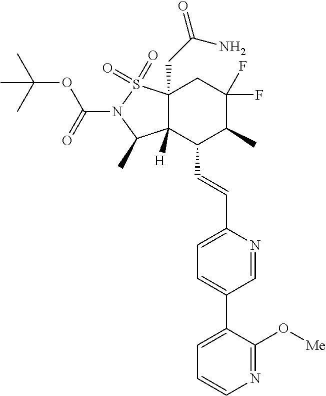 [SMILES: COc1ncccc1-c1ccc(/C=C/[C@@H]2[C@@H]3[C@@H](C)N(C(=O)OC(C)(C)C)S(=O)(=O)[C@@]3(CC(N)=O)CC(F)(F)[C@H]2C)nc1]

The reaction of Step 1 of Example 18 also yields a minor side-product, (3R,3aR,4S,5S,7aS)-tert-butyl 7a-(2-amino-2-oxoethyl)-6,6-difluoro-4-((E)-2-(2′-methoxy-[3,3′-bipyridin]-6-yl)vinyl)-3,5-dimethylhexahydrobenzo[d]isothiazole-2(3H)-carboxylate 1,1-dioxide. ¹H NMR (400 MHz, MeOD) δ 8.70 (d, J=2.0 Hz, 1H), 8.20 (dd, J=5.0, 1.8 Hz, 1H), 8.02 (dd, J=8.2, 2.3 Hz, 1H), 7.82 (dd, J=7.4, 1.8 Hz, 1H), 7.62 (d, J=8.2 Hz, 1H), 7.11 (dd, J=7.4, 5.0 Hz, 1H), 6.82 (dt, J=27.5, 12.1 Hz, 2H), 4.62 (s, 1H), 4.27 (dd, J=6.6, 3.9 Hz, 1H), 3.99 (d, J=4.2 Hz, 3H), 3.26 (d, J=16.9 Hz, 1H), 2.90 (d, J=16.7 Hz, 1H), 2.65 (ddd, J=38.1, 25.8, 8.2 Hz, 3H), 2.10 (t, J=14.8 Hz, 1H), 1.45 (d, J=15.9 Hz, 9H), 1.31 (d, J=6.9 Hz, 3H), 1.07 (d, J=6.6 Hz, 3H). LCMS (M+H)⁺ 607. PAR-1 FLIPR IC₅₀=54 nM

The following examples in Table 3 were prepared according to Scheme 3 using the procedure outlined for the synthesis of Example 18 form prepared or commercially available materials.

TABLE 3

| | | | LCMS | PAR-1 |
|---|---|---|---|---|
| Ex. | Structure | IUPAC Name | [M + H]+ | FLIPR IC₅₀ (nM) |
| 20 | 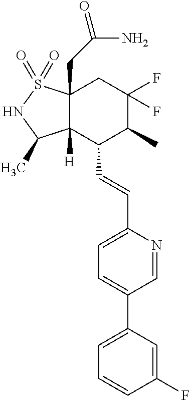 [SMILES: C[C@H]1NS(=O)(=O)[C@]2(CC(N)=O)CC(F)(F)[C@@H](C)[C@H](/C=C/c3ccc(-c4cccc(F)c4)cn3)[C@H]12] | 2-((3R,3aR,4S,5S,7aR)-6,6-difluoro-4- ((E)-2-(5-(3-fluorophenyl)pyridin-2- yl)vinyl)-3,5- dimethyl-1,1- dioxidooctahydrobenzo[d]isothiazol-7a- yl)acetamide | 494 | 5 |
| 21 | 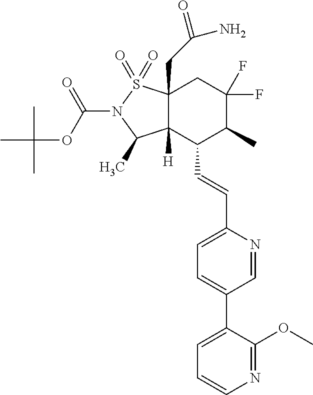 [SMILES: COc1ncccc1-c1ccc(/C=C/[C@@H]2[C@@H]3[C@@H](C)N(C(=O)OC(C)(C)C)S(=O)(=O)[C@]3(CC(N)=O)CC(F)(F)[C@H]2C)nc1] | 3R,3aR,4S,5S,7aR)-tert-butyl 7a-(2- amino-2-oxoethyl)-6,6-difluoro-4-((E)- 2-(2′-methoxy-[3,3′-bipyridin]-6- yl)vinyl)-3,5- dimethylhexahydrobenzo[d]isothiazole- 2(3H)-carboxylate 1,1-dioxide | 607 | 110 |
| 22 | 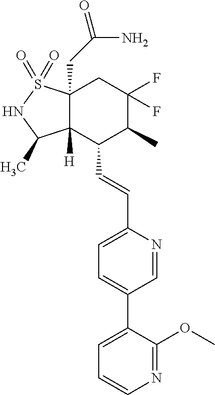 [SMILES: COc1ncccc1-c1ccc(/C=C/[C@@H]2[C@@H]3[C@@H](C)NS(=O)(=O)[C@@]3(CC(N)=O)CC(F)(F)[C@H]2C)nc1] | 2-((3R,3aR,4S,5S,7aS)-6,6-difluoro-4- ((E)-2-(2′-methoxy-[3,3′-bipyridin]-6- yl)vinyl)-3,5-dimethyl-1,1- dioxidooctahydrobenzo[d]isothiazol-7a- yl)acetamide | 507 | 337 |
| 23 | 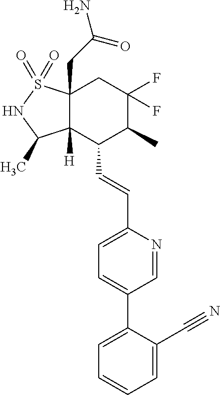 [SMILES: C[C@H]1NS(=O)(=O)[C@]2(CC(N)=O)CC(F)(F)[C@@H](C)[C@H](/C=C/c3ccc(-c4ccccc4C#N)cn3)[C@H]12] | 2-((3R,3aR,4S,5S,7aR)-4-((E)-2-(5-(2- cyanophenyl)pyridin-2-yl)vinyl)-6,6- difluoro-3,5-dimethyl-1,1- dioxidooctahydrobenzo[d]isothiazol-7a- yl)acetamide | 501 | 3 |
| 24 | 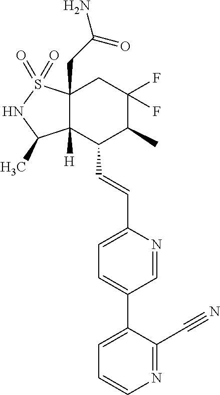 [SMILES: C[C@H]1NS(=O)(=O)[C@]2(CC(N)=O)CC(F)(F)[C@@H](C)[C@H](/C=C/c3ccc(-c4cccnc4C#N)cn3)[C@H]12] | 2-((3R,3aR,4S,5S,7aR)-6,6-difluoro-4- ((E)-2-(2′-cyano-[3,3′-bipyridin]-6- yl)vinyl)-3,5-dimethyl-1,1- dioxidooctahydrobenzo[d]isothiazol-7a- yl)acetamide | 502 | 89 |

### Example 25

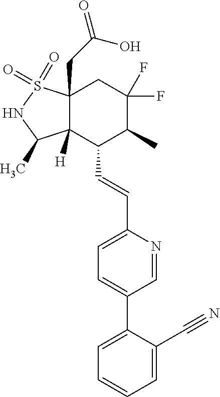 [SMILES: C[C@H]1NS(=O)(=O)[C@]2(CC(=O)O)CC(F)(F)[C@@H](C)[C@H](/C=C/c3ccc(-c4ccccc4C#N)cn3)[C@H]12]

### 2-((3R,3aR,4S,5S,7aR)-4-((E)-2-(5-(2-cyanophenyl)pyridin-2-yl)vinyl)-6,6-difluoro-3,5-dimethyl-1,1-dioxidooctahydrobenzo[d]isothiazol-7a-yl)acetic acid (Scheme 3)

### Step 1: 4-((E)-2-(5-(2-cyanophenyl)pyridin-2-yl)vinyl)-7a-(2-ethoxy-2-oxoethyl)-6,6-difluoro-3,5-dimethylhexahydrobenzo[d]isothiazole-2(3H)-carboxylate 1,1-dioxide

LDA (0.1 mL, 2M in THF, 0.2 mmol) was added dropwise to a solution of (3R,3aR,4S,5S,7aR)-tert-butyl 4-((E)-2-(5-(2-cyanophenyl)pyridin-2-yl)vinyl)-6,6-difluoro-3,5-dimethylhexahydrobenzo[d]isothiazole-2(3H)-carboxylate 1,1-dioxide (30 mg, 0.055 mmol) in de-oxygenated THF (2 mL) at −78° C. The reaction mixture was stirred at −78° C. for 1 h, then ethyl 2-bromoacetate (16 mg, 0.096 mmol) was added and the reaction mixture was stirred at −78° C. for another 2 h. The reaction mixture was quenched with saturated aqueous NH₄Cl (1 mL) and was warmed up to room temperature and further diluted with EtOAc (20 mL) and water (20 mL). The aqueous layer was extracted with EtOAc (20 mL) and the combined organic layers were dried over anhydrous Na₂SO₄, concentrated and purified by prep-TLC (EtOAc/hexanes=1/2) to afford the title compound. ¹H NMR (400 MHz, CDCl₃-d₁) δ 8.72 (s, 1H), 7.94 (d, J=6.3 Hz, 1H), 7.82 (d, J=7.8 Hz, 1H), 7.73-7.69 (m, 1H), 7.56-7.51 (m, 2H), 7.34 (d, J=8.0 Hz, 1H), 6.70 (s, 2H), 4.25-4.18 (m, 3H), 3.16-3.02 (m, 2H), 3.02-2.90 (m, 1H), 2.82-2.74 (m, 1H), 2.44-2.37 (m, 1H), 2.25-2.02 (m, 2H), 1.54 (s, 9H), 1.43-1.40 (m, 3H), 1.33-1.29 (m, 3H), 1.11-1.07 (m, 3H). LCMS (M+H)⁺ 630. PAR-1 FLIPR IC₅₀=54 nM

### Step 2: 2-((3R,3aR,4S,5S,7aR)-4-((E)-2-(5-(2-cyanophenyl)pyridin-2-yl)vinyl)-6,6-difluoro-3,5-dimethyl-1,1-dioxidooctahydrobenzo[d]isothiazol-7a-yl)acetic acid

A solution of 4-((E)-2-(5-(2-cyanophenyl)pyridin-2-yl)vinyl)-7a-(2-ethoxy-2-oxoethyl)-6,6-difluoro-3,5-dimethylhexahydrobenzo[d]isothiazole-2(3H)-carboxylate 1,1-dioxide (12 mg, 0.038 mmol) in hydrochloric acid (4 M in dioxanes, 1 mL) and MeCN (4 mL) was stirred at 40° C. for 60 h. The solvent was removed under reduced pressure. The residue was purified by HPLC (Acetonitrile/Water+0.0 1% TFA modifier) to afford the title compound. ¹H NMR (400 MHz, MeOD) δ 8.63 (s, 1H), 7.96 (d, J=9.8 Hz, 1H), 7.80 (d, J=7.7 Hz, 1H), 7.70 (t, J=7.6 Hz, 1H), 7.63-7.47 (m, 3H), 6.79-6.59 (m, 2H), 3.71-3.57 (m, 1H), 3.05-2.81 (m, 3H), 2.78-2.65 (m, 1H), 2.30-2.05 (m, 3H), 1.16 (d, J=6.0 Hz, 3H), 0.97 (d, J=6.4 Hz, 3H). PAR-1 FLIPR IC₅₀=12 nM

### Example 26

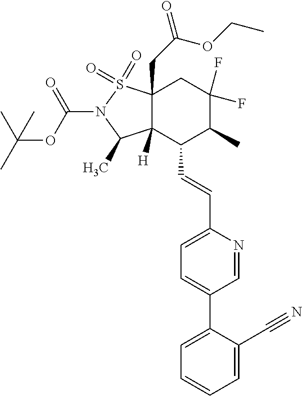 [SMILES: CCOC(=O)C[C@@]12CC(F)(F)[C@@H](C)[C@H](/C=C/c3ccc(-c4ccccc4C#N)cn3)[C@@H]1[C@@H](C)N(C(=O)OC(C)(C)C)S2(=O)=O]

### 4-((E)-2-(5-(2-cyanophenyl)pyridin-2-yl)vinyl)-7a-(2-ethoxy-2-oxoethyl)-6,6-difluoro-3,5-dimethylhexahydrobenzo[d]isothiazole-2(3H)-carboxylate 1,1-dioxide

LDA (0.1 mL, 2M in THF, 0.2 mmol) was added dropwise to a solution of (3R,3aR,4S,5S,7aR)-tert-butyl 4-((E)-2-(5-(2-cyanophenyl)pyridin-2-yl)vinyl)-6,6-difluoro-3,5-dimethylhexahydrobenzo[d]isothiazole-2(3H)-carboxylate 1,1-dioxide (30 mg, 0.055 mmol) in de-oxygenated THF (2 mL) at −78° C. The reaction mixture was stirred at −78° C. for 1 h, then ethyl 2-bromoacetate (16 mg, 0.096 mmol) was added and the reaction mixture was stirred at −78° C. for another 2 h. The reaction mixture was quenched with saturated aqueous NH₄Cl (1 mL) and was warmed up to room temperature and further diluted with EtOAc (20 mL) and water (20 mL). The aqueous layer was extracted with EtOAc (20 mL) and the combined organic layers were dried over anhydrous Na₂SO₄, concentrated and purified by prep-TLC (EtOAc/hexanes=1/2) to afford the title compound. ¹H NMR (400 MHz, CDCl₃-d₁) δ 8.72 (s, 1H), 7.94 (d, J=6.3 Hz, 1H), 7.82 (d, J=7.8 Hz, 1H), 7.73-7.69 (m, 1H), 7.56-7.51 (m, 2H), 7.34 (d, J=8.0 Hz, 1H), 6.70 (s, 2H), 4.25-4.18 (m, 3H), 3.16-3.02 (m, 2H), 3.02-2.90 (m, 1H), 2.82-2.74 (m, 1H), 2.44-2.37 (m, 1H), 2.25-2.02 (m, 2H), 1.54 (s, 9H), 1.43-1.40 (m, 3H), 1.33-1.29 (m, 3H), 1.11-1.07 (m, 3H). LCMS (M+H)⁺ 630. PAR-1 FLIPR IC₅₀=54 nM

### Example 27

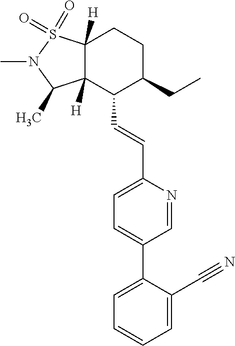 [SMILES: CC[C@@H]1CC[C@@H]2[C@H]([C@H]1/C=C/c1ccc(-c3ccccc3C#N)cn1)[C@@H](C)N(C)S2(=O)=O]

### 2-(6-((E)-2-((3R,3aR,4R,5R,7aR)-5-ethyl-2,3-dimethyl-1,1-dioxidooctahydrobenzo[d]isothiazol-4-yl)vinyl)pyridin-3-yl)benzonitrile (Scheme 4)

NaH (5 mg, 0.12 mmol, 60 wt %) was added to a solution of 2-(6-((E)-2-((3R,3aR,4R,5R,7aR)-5-ethyl-3-methyl-1,1-dioxidooctahydrobenzo[d]isothiazol-4-yl)vinyl)pyridin-3-yl)benzonitrile (5 mg, 0.012 mmol) in THF (0.5 mL) at room temperature. After stirring for 25 min at room temperature, MeI (8.4 mg, 0.06 mmol) was added and reaction was stirred for 6 h. Methanol (0.3 mL) was added to quench the reaction and volatiles were removed under reduced pressure. The residue was purified by HPLC (MeCN/water+0.08% NH₄HCO₃ modifier) to afford the title compound. ¹H NMR (400 MHz, CDCl₃)¹H NMR (400 MHz, CDCl₃) δ 8.71 (d, 1H, J=2.4 Hz), 7.90 (dd, 1H, J=2.4, 8.0 Hz), 7.81 (d, 1H, J=8.0 Hz), 7.72-7.68 (m, 1H), 7.56-7.48 (m, 2H), 7.32 (d, 1H, J=7.6 Hz), 6.80-6.55 (m, 2H), 3.50-3.40 (m, 1H), 3.20-3.10 (m, 1H), 2.76 (s, 3H), 2.70-2.62 (m, 1H), 2.50-2.40 (m, 1H), 2.25-2.15 (m, 1H), 2.05-1.95 (m, 1H), 1.65-1.55 (m, 1H), 1.50-1.35 (m, 2H), 1.32 (d, 3H, J=6.0 Hz), 1.15-0.90 (m, 2H), 0.88 (t, 3H, J=7.6 Hz). LCMS (M+H)⁺ 436. PAR-1 FLIPR IC₅₀=13 nM

The following examples in Table 4 were prepared according to Scheme 4 using the procedure outlined for the synthesis of Example 27 from prepared or commercially available materials.

TABLE 4

| | | | LCMS | PAR-1 |
|---|---|---|---|---|
| Ex. | Structure | IUPAC Name | [M + H]+ | FLIPR IC₅₀ (nM) |
| 28 | 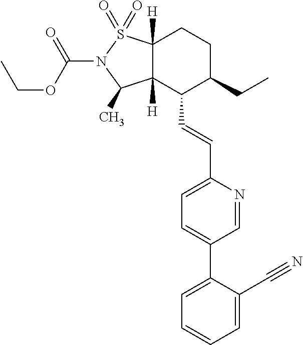 [SMILES: CCOC(=O)N1[C@H](C)[C@H]2[C@@H](/C=C/c3ccc(-c4ccccc4C#N)cn3)[C@H](CC)CC[C@H]2S1(=O)=O] | (3R,3aR,4R,5R,7aR)-ethyl 4-((E)-2-(5- (2-cyanophenyl)pyridin-2-yl)vinyl)-5- ethyl-3- methylhexahydrobenzo[d]isothiazole- 2(3H)-carboxylate 1,1-dioxide | 494 | 325 |
| 29 | 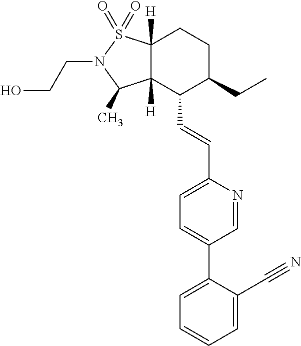 [SMILES: CC[C@@H]1CC[C@@H]2[C@H]([C@H]1/C=C/c1ccc(-c3ccccc3C#N)cn1)[C@@H](C)N(CCO)S2(=O)=O] | 2-(6-((E)-2-((3R,3aR,4R,5R,7aR)-5- ethyl-2-(2-hydroxyethyl)-3-methyl-1,1- dioxidooctahydrobenzo[d]isothiazol-4- yl)vinyl)pyridin-3-yl)benzonitrile | 466 | 540 |

### ASSAYS

The following assay was used to determine the ability of the inventive compounds to inhibit the PAR-1 receptor.

### PAR-1 FLIPR Assay

HEK 293 Cells are grown in media containing DMEM, 10% FBS pen/strep/L-Glutamine and non-essential amino acids. The cells are plated in 384-well PDL coated plates at 12000 cells/well and incubated overnight at 37° C./5% CO₂. Media is then removed from the cells, which are then incubated with buffer (Hank's containing HEPES and Chaps) containing FLIPR calcium-5 dye, made with buffer containing probenecid, for 60 minutes at 37° C. Varying concentrations of compound in a final concentration of 5% DMSO are then added to the cells and incubated at 25 degrees for 30 minutes. The plates are then added to a FLIPR Tetra cellular screening system and the device adds a concentration of a PAR1 selective receptor-activating peptide with the sequence Ala-parafluoroPhe-Arg-Cha-Cit-Tyr-NH₂ (SEQ ID NO: 3) (prepared in water) at a concentration equal to the effective concentration that achieves 80% activation of signaling on the day of the experiment. The range is from 1.4-2.0 uM peptide. The FLIPR reads at an excitation wavelength of 480 nm and an emission wavelength of 535 nm, and performs 60 scans over a 1-2 min reading time. The data are analyzed by taking the peak signal over a portion of the range of the 60 scans and dividing this signal by the minimum signal for that same range. The data are expressed as percent inhibition of the maximum divided by the minimum signal achieved at 80% activation produced by the PAR1 activating peptide on the test day.

While the invention has been described with reference to certain particular embodiments thereof, numerous alternative embodiments will be apparent to those skilled in the art from the teachings described herein. Recitation or depiction of a specific compound in the claims (i.e., a species) without a specific stereoconfiguration designation, or with such a designation for less than all chiral centers, is intended to encompass for the non-specified sterocenters the racemate, racemic mixtures, each individual enantiomer, a diastereoisomeric mixture and each individual diastereomer of the compound where such forms are possible due to the presence of one or more asymmetric centers. All patents, patent applications and publications cited herein are incorporated by reference in their entirety.

1. A compound of Formula I 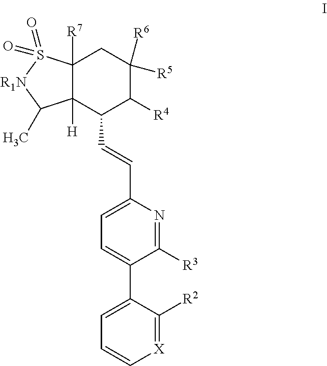
or a pharmaceutically acceptable salt thereof, wherein:
X is —N—, —CH or —CR⁸;
R¹ is selected from the group consisting of hydrogen, C₁-C₆alkyl, COC₁-C₆alkyl, COOH and COOC₁-C₆alkyl, wherein any C₁-C₆alkyl can be unsubstituted or substituted with one or more substituents independently selected from the group consisting of halo, —OH and —CN;
R² is selected from the group consisting of halo, C₁-C₆alkyl, OC₁-C₆alkyl, —CN and hydrogen, wherein when X is —CR⁸, R² is hydrogen;
R³ is selected from the group consisting of hydrogen, halo and C₁-C₆alkyl;
R⁴ is selected from the group consisting of hydrogen, halo and C₁-C₆alkyl;
R⁵ is selected from the group consisting of hydrogen, halo and C₁-C₆alkyl;
R⁶ is selected from the group consisting of hydrogen, halo and C₁-C₆alkyl;
R⁷ is selected from the group consisting of hydrogen and C₁-C₆alkyl, wherein C₁-C₆alkyl can be unsubstituted or substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN; and
R⁸ is selected from the group consisting of halo and C₁-C₆alkyl.

2. The compound of Formula I of claim 1, having the following stereochemistry as shown in a compound of Formula Ia 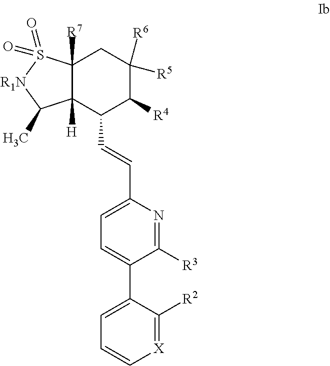
or a pharmaceutically acceptable salt thereof, wherein:
X is —N—, —CH or —CR⁸;
R¹ is selected from the group consisting of hydrogen, C₁-C₆alkyl, COC₁-C₆alkyl, COOH and COOC₁-C₆alkyl, wherein any C₁-C₆alkyl can be unsubstituted or substituted with one or more substituents independently selected from the group consisting of halo, —OH and —CN;
R² is selected from the group consisting of halo, C₁-C₆alkyl, OC₁-C₆alkyl, —CN and hydrogen wherein when X is —CR⁸, R² is hydrogen;
R³ is selected from the group consisting of hydrogen, halo and C₁-C₆alkyl;
R⁴ is selected from the group consisting of hydrogen, halo and C₁-C₆alkyl;
R⁵ is selected from the group consisting of hydrogen, halo and C₁-C₆alkyl;
R⁶ is selected from the group consisting of hydrogen, halo and C₁-C₆alkyl;
R⁷ is selected from the group consisting of hydrogen and C₁-C₆alkyl, wherein C₁-C₆alkyl can be unsubstituted or substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN; and
R⁸ is selected from the group consisting of halo and C₁-C₆alkyl.

3. The compound of Formula I of claim 1, having the following stereochemistry as shown in a compound of Formula Ib: 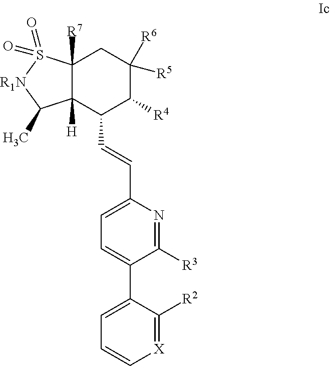
or a pharmaceutically acceptable salt thereof, wherein:
X is —N—, —CH or —CR⁸;
R¹ is selected from the group consisting of hydrogen, C₁-C₆alkyl, COC₁-C₆alkyl, COOH and COOC₁-C₆alkyl, wherein any C₁-C₆alkyl can be unsubstituted or substituted with one or more substituents independently selected from the group consisting of halo, —OH and —CN;
R² is selected from the group consisting of halo, C₁-C₆alkyl, OC₁-C₆alkyl, —CN and hydrogen wherein, when X is —CR⁸, R² is hydrogen;
R³ is selected from the group consisting of hydrogen, halo and C₁-C₆alkyl;
R⁴ is selected from the group consisting of hydrogen, halo and C₁-C₆alkyl;
R⁵ is selected from the group consisting of hydrogen, halo and C₁-C₆alkyl;
R⁶ is selected from the group consisting of hydrogen, halo and C₁-C₆alkyl;
R⁷ is selected from the group consisting of hydrogen and C₁-C₆alkyl, wherein C₁-C₆alkyl can be unsubstituted or substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN; and
R⁸ is selected from the group consisting of halo and C₁-C₆alkyl.

4. The compound of claim 1, or a pharmaceutically acceptable salt thereof, wherein X is —N—.

5. The compound of claim 1, or a pharmaceutically acceptable salt thereof, wherein X is —CH—.

6. The compound of claim 1, or a pharmaceutically acceptable salt thereof, wherein X is —CR⁸ and R² is hydrogen.

7. The compound of claim 1, or a pharmaceutically acceptable salt thereof, wherein R¹ is hydrogen or COOC₁-C₆alkyl.

8. The compound of claim 1, or a pharmaceutically acceptable salt thereof, wherein R² is selected from the group consisting of methoxy or —CN.

9. The compound of claim 1, or a pharmaceutically acceptable salt thereof, wherein R³ is selected from the group consisting of hydrogen or methyl.

10. The compound of claim 1, or a pharmaceutically acceptable salt thereof, wherein R⁴ is selected from the group consisting of hydrogen, methyl or ethyl.

11. The compound of claim 1, or a pharmaceutically acceptable salt thereof, wherein R⁵ is selected from the group consisting of hydrogen or fluoro.

12. The compound of claim 1, or a pharmaceutically acceptable salt thereof, wherein R⁶ is selected from the group consisting of hydrogen or fluoro.

13. The compound of claim 1, or a pharmaceutically acceptable salt thereof, wherein R⁷ is selected from the group consisting of hydrogen, methyl or ethyl.

14. The compound of claim 1, or a pharmaceutically acceptable salt thereof, wherein R⁸ is fluoro.

15. The compound of claim 1, or a pharmaceutically acceptable salt thereof, selected from the group consisting of:

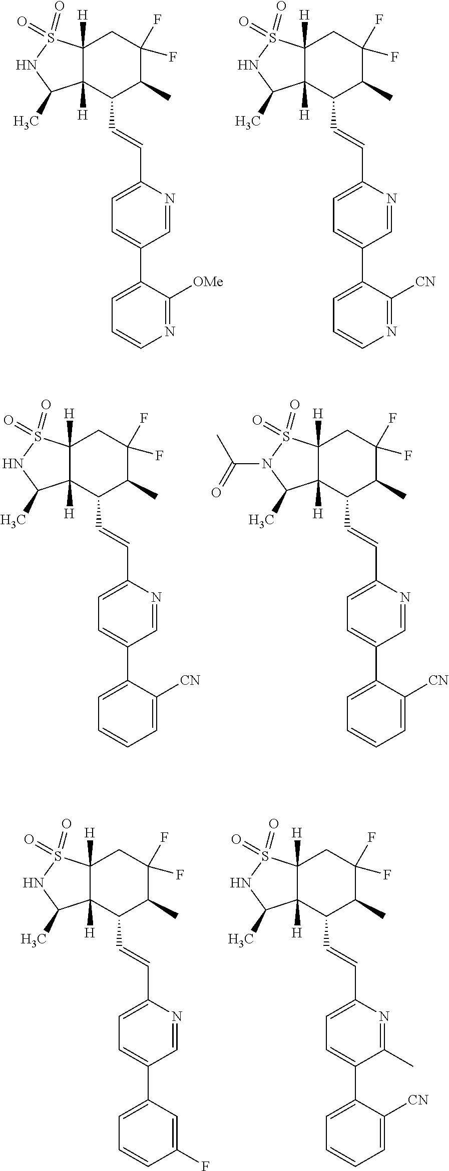

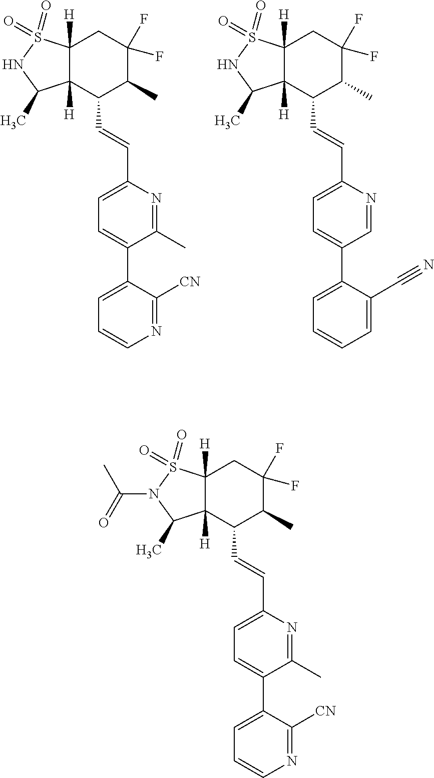 [SMILES: Cc1nc(/C=C/[C@@H]2[C@H]3[C@@H](CC(F)(F)[C@H]2C)S(=O)(=O)N[C@@H]3C)ccc1-c1cccnc1C#N ; C[C@@H]1[C@H](/C=C/c2ccc(-c3ccccc3C#N)cn2)[C@H]2[C@@H](CC1(F)F)S(=O)(=O)N[C@@H]2C ; CC(=O)N1[C@H](C)[C@H]2[C@@H](/C=C/c3ccc(-c4cccnc4C#N)c(C)n3)[C@H](C)C(F)(F)C[C@H]2S1(=O)=O]

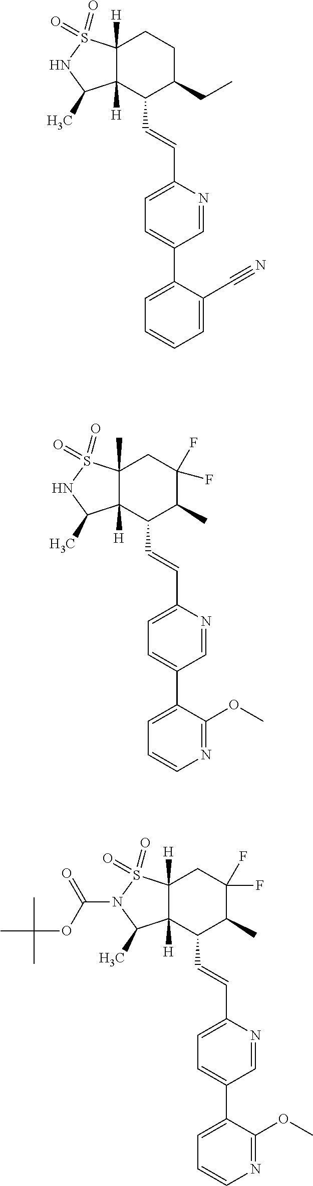

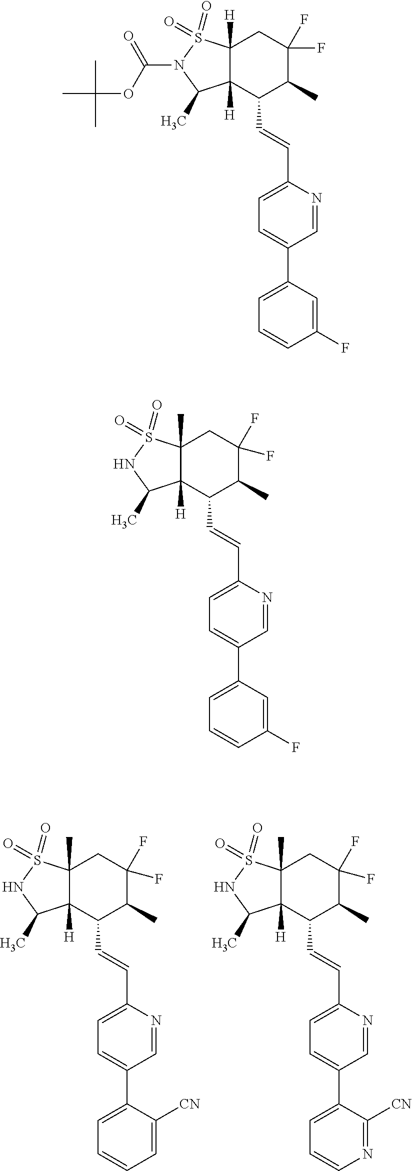 [SMILES: C[C@@H]1[C@H]2[C@@H](/C=C/c3ccc(-c4cccc(F)c4)cn3)[C@H](C)C(F)(F)C[C@H]2S(=O)(=O)N1C(=O)OC(C)(C)C ; C[C@H]1NS(=O)(=O)[C@]2(C)CC(F)(F)[C@@H](C)[C@H](/C=C/c3ccc(-c4cccc(F)c4)cn3)[C@H]12 ; C[C@H]1NS(=O)(=O)[C@]2(C)CC(F)(F)[C@@H](C)[C@H](/C=C/c3ccc(-c4ccccc4C#N)cn3)[C@H]12 ; C[C@H]1NS(=O)(=O)[C@]2(C)CC(F)(F)[C@@H](C)[C@H](/C=C/c3ccc(-c4cccnc4C#N)cn3)[C@H]12]

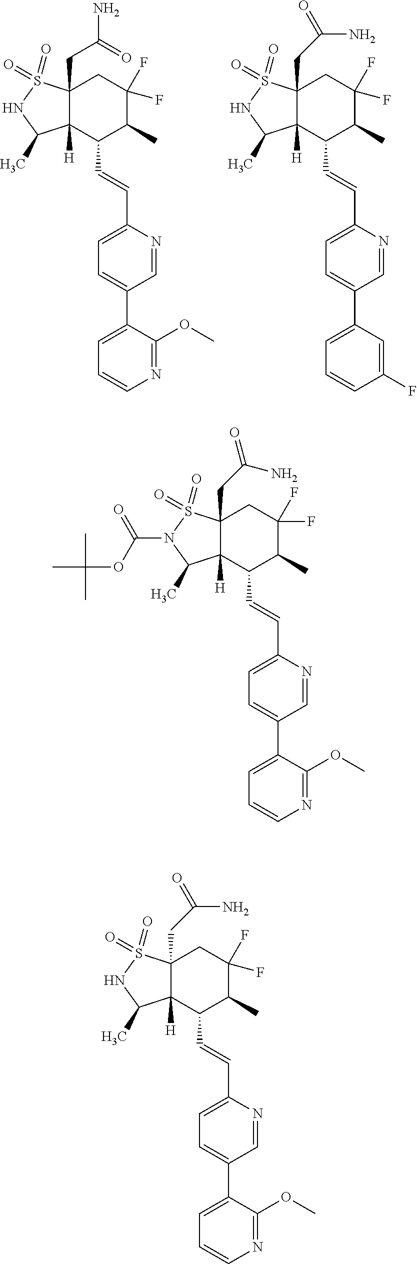 [SMILES: COc1ncccc1-c1ccc(/C=C/[C@@H]2[C@@H]3[C@@H](C)NS(=O)(=O)[C@]3(CC(N)=O)CC(F)(F)[C@H]2C)nc1 ; C[C@H]1NS(=O)(=O)[C@]2(CC(N)=O)CC(F)(F)[C@@H](C)[C@H](/C=C/c3ccc(-c4cccc(F)c4)cn3)[C@H]12 ; COc1ncccc1-c1ccc(/C=C/[C@@H]2[C@@H]3[C@@H](C)N(C(=O)OC(C)(C)C)S(=O)(=O)[C@]3(CC(N)=O)CC(F)(F)[C@H]2C)nc1 ; COc1ncccc1-c1ccc(/C=C/[C@@H]2[C@@H]3[C@@H](C)NS(=O)(=O)[C@@]3(CC(N)=O)CC(F)(F)[C@H]2C)nc1]

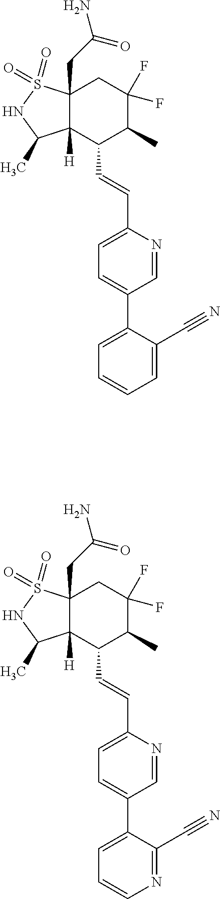 [SMILES: C[C@H]1NS(=O)(=O)[C@]2(CC(N)=O)CC(F)(F)[C@@H](C)[C@H](/C=C/c3ccc(-c4ccccc4C#N)cn3)[C@H]12 ; C[C@H]1NS(=O)(=O)[C@]2(CC(N)=O)CC(F)(F)[C@@H](C)[C@H](/C=C/c3ccc(-c4cccnc4C#N)cn3)[C@H]12]

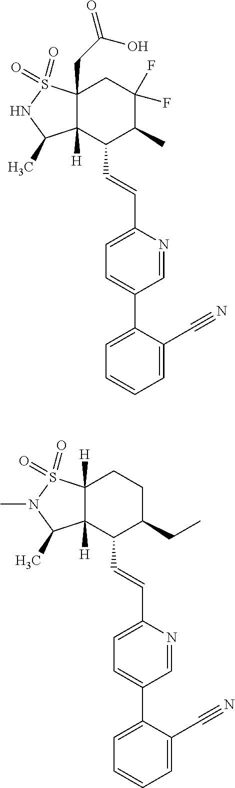 [SMILES: C[C@H]1NS(=O)(=O)[C@]2(CC(=O)O)CC(F)(F)[C@@H](C)[C@H](/C=C/c3ccc(-c4ccccc4C#N)cn3)[C@H]12 ; CC[C@@H]1CC[C@@H]2[C@H]([C@H]1/C=C/c1ccc(-c3ccccc3C#N)cn1)[C@@H](C)N(C)S2(=O)=O]

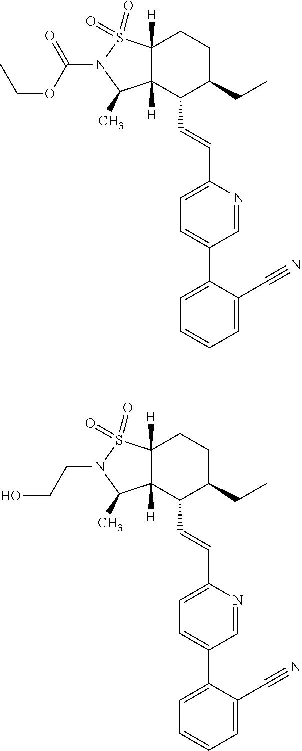 [SMILES: CCOC(=O)N1[C@H](C)[C@H]2[C@@H](/C=C/c3ccc(-c4ccccc4C#N)cn3)[C@H](CC)CC[C@H]2S1(=O)=O ; CC[C@@H]1CC[C@@H]2[C@H]([C@H]1/C=C/c1ccc(-c3ccccc3C#N)cn1)[C@@H](C)N(CCO)S2(=O)=O]

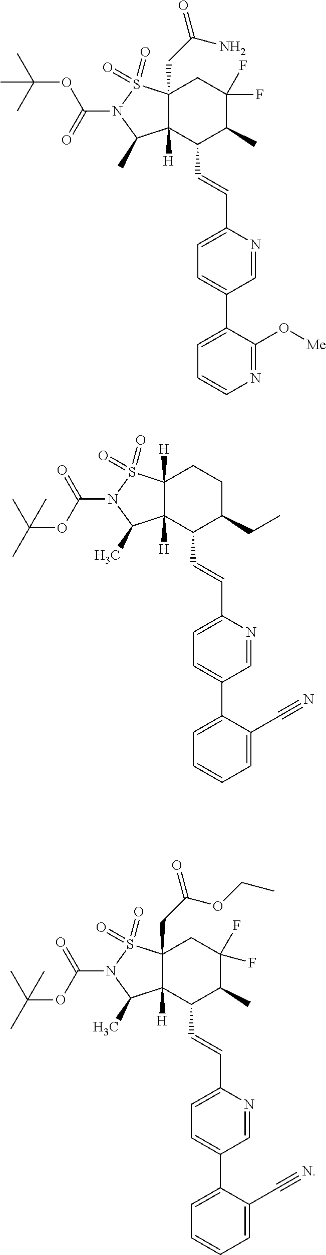 [SMILES: COc1ncccc1-c1ccc(/C=C/[C@@H]2[C@@H]3[C@@H](C)N(C(=O)OC(C)(C)C)S(=O)(=O)[C@@]3(CC(N)=O)CC(F)(F)[C@H]2C)nc1 ; CC[C@@H]1CC[C@@H]2[C@H]([C@H]1/C=C/c1ccc(-c3ccccc3C#N)cn1)[C@@H](C)N(C(=O)OC(C)(C)C)S2(=O)=O ; CCOC(=O)C[C@@]12CC(F)(F)[C@@H](C)[C@H](/C=C/c3ccc(-c4ccccc4C#N)cn3)[C@@H]1[C@@H](C)N(C(=O)OC(C)(C)C)S2(=O)=O]

16. A pharmaceutical composition comprising an effective amount of a compound as defined in of claim 1, or a pharmaceutically acceptable salt thereof and a pharmaceutically acceptable carrier.

17. A method for treating acute coronary syndrome, or for secondary prevention of myocardial infarction or stroke or peripheral artery disease by administering at least one compound of Formula I: 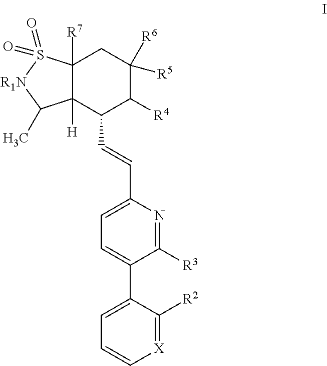
or a pharmaceutically acceptable salt thereof, wherein:
X is —N—, —CH or —CR⁸;
R¹ is selected from the group consisting of hydrogen, C₁-C₆alkyl, COC₁-C₆alkyl, COOH and COOC₁-C₆alkyl, wherein any C₁-C₆alkyl can be unsubstituted or substituted with one or more substituents independently selected from the group consisting of halo, —OH and —CN;
R² is selected from the group consisting of halo, C₁-C₆alkyl, OC₁-C₆alkyl, —CN and hydrogen wherein when X is —CR⁸, R² is hydrogen;
R³ is selected from the group consisting of hydrogen, halo and C₁-C₆alkyl;
R⁴ is selected from the group consisting of hydrogen, halo and C₁-C₆alkyl;
R⁵ is selected from the group consisting of hydrogen, halo and C₁-C₆alkyl;
R⁶ is selected from the group consisting of hydrogen, halo and C₁-C₆alkyl;
R⁷ is selected from the group consisting of hydrogen and C₁-C₆alkyl, wherein C₁-C₆alkyl can be unsubstituted or substituted with one or more substituents independently selected from the group consisting of CONH₂, CONHC₁-C₆alkyl, CON(C₁-C₆alkyl)₂, COC₁-C₆alkyl, COOH, COOC₁-C₆alkyl, halo, —OH and —CN; and
R⁸ is selected from the group consisting of halo and C₁-C₆alkyl, to a mammal in need of such treatment.


SEQUENCE LISTING (3 sequences)

SEQ ID NO: 1 - protein, 4 aa, Artificial Sequence
  thrombin receptor antagonist peptide
  N-transcinnamoyl-p-fluoroPhe (1)..(1)
  p-guanidinoPhe (2)..(2)
XXLR

SEQ ID NO: 2 - protein, 5 aa, Artificial Sequence
  thrombin receptor antagonist peptide
  N-transcinnamoyl-p-fluoroPhe (1)..(1)
  p-guanidinoPhe (2)..(2)
XXLRR

SEQ ID NO: 3 - protein, 6 aa, Artificial Sequence
  PAR1 selective receptor-activating peptide
  parafluoroPhe (2)..(2)
  beta-cyclohexylalanine (4)..(4)
  citrulline (5)..(5)
AXRXXY